SEQUENCE LISTING (84 sequences)

SEQ ID NO: 1 - DNA, 2057 bp, Homo sapiens
  misc_feature (1)...(2057): n = A,T,C or G
ggatcctcggcggcggcggcggtgcttacagcctgagaagagcgtctcgcccnggagcggcggcggccatcgagacccacccaaggcgcgtccccctcggcctcccagcgctcccaagcygcgrccgcgccccttcagctagctcgctcgctcgctctgcttccctgctgccggctgcgcatggcgttggcgttggcggcgctggcggcggtcgagccggcctgcggcagccggtaccagcagttgcagaatgaagaagagtctggagaacctgaacaggctgcaggtgatgctcctccaccttacagcagcatttctgcagagagcgcassatattttgactacaaggatgagtctgggtttccaaagcccccatcttacaatgtagctacaacactgcccagttatgatgaagcggagaggaccaaggctgaagctactatccctttggttcctgggagagatgaggattttgtgggtcgggatgattttgatgatgctgaccagctgaggataggaaatgatgggattttcatgttaacttttttcatggcattcctctttaactggattgggtttttcctgtctttttgcctgaccacttcagctgcaggaaggtatggggccatttcaggatttggtctctctctaattaaatggatcctgattgtcaggttttccacctatttccctggatattttgatggtcagtactggctctggtgggtgttccttgttttaggctttctcctgtttctcagaggatttatcaattatgcaaaagttcggaagatgccagaaactttctcaaatctccccaggaccagagttctctttatttattaaagatgttttctggcaaaggccttcctgcatttatgaattctctctcaagaagcaagagaacacctgcaggaagtgaatcaagatgcagaacacagaggaataatcacctgctttaaaaaaataaagtactgttgaaaagatcatttctctctatttgttcctaggtgtaaaattttaatagttaatgcagaattctgtaatcattgaatcattagtggttaatgtttgaaaaagctcttgcaatcaagtctgtgatgtattaataatgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgttatgactgtgtttttgcacataatccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctatgttaaaactgtaaatacaacagaacattaataaatatctcttgtgtagcaccttttaaaaaaaaaaaaaaaaaaaaamwvgcctgccattttggttgattgccnaggcaaattttttccnaatcctangggaatcnttccaggtagatgcgattaaaaaanccaatggtnggggccaatttttttcctccattttcaggcnccaagaaggcccc

SEQ ID NO: 2 - DNA, 725 bp, Homo sapiens
tttttttttttcattttttaacaagcaaattttaataatgccttttatttctatacaaagcaatgtaactttctgaaaaaaaaaatggctatacagaaccctttaaacataagagtacagagtttcaaatggcaacaagaagttaagaaacatagggcactgtgtcgttatgggtgaatcctagtcgtcctgcagcccaaggtccaagctagtttactccataaccttaagtaaataaccgcggttcctatgaatacctttccaaaaccatttattataaaaactcactctttatccattatcagtattaacgtatcaaagtgagaaagaacatgggagagaaaaatgacaattaatgcagcgtgaacattttcatcctgaataccagtaaggactctagatgatatatggtgcccacttcatgtgcctcaaaagattttaattttcagaacatttaaacgattaaagagaaatcatcctcttaactaggtattttaaagcktatcaaactgtaggtttacatttgacaaagtcttatggaccaccaaatacgttrgaawtaggaattccaaatatttcagttcgttgaatatacaaaaccaaataaggcgcaaggacctgaagaataaacacaaagctagtctacaatgtttgagttaacgtgatttaggacccttctattttgttctgcaagataatttacatagttctctctgctatatgtg

SEQ ID NO: 3 - DNA, 3599 bp, Homo sapiens
gcggcggctgcggtgcttacagcctgagaagagcgtctcgcccgggagcggcggcggcggtgcttacagcctgagaagagcgtctcgcccgggagcggcggcggccatcgagacccacccaaggcgcgtccccctcggcctcccagcgctcccaagccgcagcggccgcgccccttcagctagctcgctcgctcgctctgcttccctgctgccggctgcgccatggcgttggcgttggcggcgctggcggcggtcgagccggcctgcggcagccggtaccagcagttgcagaatgaagaagagtctggagaacctgaacaggctgcaggtgatgctcctccaccttacagcagcatttctgcagagagcgcagcatattttgactacaaggatgagtctgggtttccaaagcccccatcttacaatgtagctacaacactgcccagttatgatgaagcggagaggaccaaggctgaagctactatccctttggttcctgggagagatgaggattttgtgggtcgggatgattttgatgatgctgaccagctgaggataggaaatgatgggattttcatgttaacttttttcatggcattcctctttaactggattgggtttttcctgtctttttgcctgaccacttcagctgcaggaaggtatggggccatttcaggatttggtctctctctaattaaatggatcctgattgtcaggttttccacctatttccctggatattttgatggtcagtactggctctggtgggtgttccttgttttaggctttctcctgtttctcagaggatttatcaattatgcaaaagttcggaagatgccagaaactttctcaaatctccccaggaccagagttctctttatttattaaagatgttttctggcaaaggccttcctgcatttatgaattctctctcaagaagcaagagaacacctgcaggaagtgaatcaagatgcagaacacagaggaataatcacctgctttaaaaaaataaagtactgttgaaaagatcatttctctctatttgttcctaggtgtaaaattttaatagttaatgcagaattctgtaatcattgaatcattagtggttaatgtttgaaaaagctcttgcaatcaagtctgtgatgtattaataatgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgttatgactgtgtttttgcacataatccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctatgttaaaactgtaaatacaacagaacattaataaatatctcttgtgtagcaccttttactgtagattagtgcttaatttcttggcttgcatttgttgattgctaaggcaattttttctaatcttagggaatcattcagtagatgcgattaaaaaactaatgttgggtcaatttttttcttcattttcagcacaagaagtcctcttatatcctactaaatacattcctaaaaatgtatttgaacattggttctgtaaaagataatggactaaaaaagtagagaggagttgtagagatcttaaatcattctggaattcctaattatgcttcaatttttagacataattttagataatttatttccagtgttttctgcatgttctcatttgttctttttctcagttgaatgcaccaactggtttgagtcctgtgagcattcagtcagttgaaattaaagattcctcatttctcctgatttctattcttgtctcaatcttaaatttagagaccagttgtttttatgatatcagccatttgatttttttcattttctatttaagaaatatgaagaaaaaatacaccaagatggtcaaattactacacaaatcagcaccagcacagtctgatagctgcaaatgtccattcatctgctgtgtatgtatatccagaatcagcataggaagtcgttcaggatatcagtatataatgcacagaagtgtgggttgtttgaaagccaaacaggaaaattaggagcctcctggattgacatttcagtgatccctctaaccagtttatggattattatgaataatagtgtagtgtgttctttttcagaagttatatttgataatagagaagggagttttatggaagtttctttgaagatttttttttttccatttcgaatcagattatagcaacaatggagtttggaagtttgtatggcctataatgttctaagttccagaatgaaaagatctgtaacaatctgaatagatgtggacacatatagcagagagaactatgtaaattatcttgcagaacaaaatagaagggtcctaaatcacgttaactcaaacattgtagactagctttgtgtttattcttcaggtccttgcgccttatttggttttgtatattcaacgaactgaaatatttggaattcctatttctacgtatttggtggtccataagactttgtcaaatgtaaacctacagtttgataagctttaaaatacctagttaagaggatgatttctctttaatcgtttaaatgttctgaaaattaaaatcttttgaggcacatgaagtgggcaccatatatcatctagagtccttactggtattcaggatgaaaatgttcacgctgcattaattgtcatttttctctcccatgttctttctcactttgatacgttaatactgataatggataaagagtgagtttttataataaatggttttggaaaggtattcataggaaccgcggttatttacttaaggttatggagtaaactagcttggaccttgggctgcaggacgactaggattcacccataacgacacagtgccctatgtttcttaacttcttgttgccatttgaaactctgtactcttatgtttaaagggttctgtatagccattttttttttcagaaagttacattgctttgtatagaaataaaaggcattattaaaatttgcttgttaaaaaatg

SEQ ID NO: 4 - protein, 221 aa, Homo sapiens
MALALAALAAVEPACGSRYQQLQNEEESGEPEQAAGDAPPPYSSISAESAAYFDYKDESGFPKPPSYNVATTLPSYDEAERTKAEATIPLVPGRDEDFVGRDDFDDADQLRIGNDGIFMLTFFMAFLFNWIGFFLSFCLTTSAAGRYGAISGFGLSLIKWILIVRFSTYFPGYFDGQYWLWWVFLVLGFLLFLRGFINYAKVRKMPETFSNLPRTRVLFIY

SEQ ID NO: 5 - DNA, 1054 bp, Homo sapiens
gcaaaggccttcctgcatttatgaattctctctcaagaagcaagagaacacctgcaggaagtgaatcaagatgcagaacacagaggaataatcacctgctttaaaaaaataaagtactgttgaaaagatcatttctctctatttgttcctaggtgtaaaattttaatagttaatgcagaattctgtaatcattgaatcattagtggttaatgtttgaaaaagctcttgcaatcaagtctgtgatgtattaataatgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgttatgactgtgtttttgcacataatccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctatgttaaaactgtaaatacaacagaacattaataaatatctcttgtgtagcaaaaaaaaaaaaaaaaaaaa

SEQ ID NO: 6 - DNA, 50000 bp, Homo sapiens
cttcaccccttagtagctctgtgtcattgggtgagatatttaatttctttgggcataacttcctcatctgtgaaacactgataattgtatctcctatatggcagctatgccagcctcctttctgttcttccacaagacaatctctctctggaccgccctttgcacctgttcctgtggtcgggaaagcctttagcatggccaaacccttcaagttttgacttcatgtcaccgagtcagggagcttttttggaaaatattgtttccgattccgcagatgaggctgttaaaatactaaaggatggcataaacatgaggatgctattataatcatccaccctaagtaggatttgagatccaagcagttctcaaggatttagggttagcaaactttttctgtaaaatgtcagatagcaagtattatagggtttgcaggccataccaaacaaatgggtgctgctgtgttccaataaaactttatttatggacactgaaacttgaatttcatgtacttttcacagtcatggtggcagaccggatttggccccctccctgtcgctggctgacccctggtttatgagtacggcagataaaagaagggagtgaataagaaccacgattctgctgccagacacgagggatcaggtcgctgctgtgaagattgtcaacctctctggttgccaacgacctcgagaatggggttgtggtccctgcgtcagggcggttgtgctgaaggctggatgagggcacgtaggtgaagctcagtgcttggcacgaggtgctcatcagcagcatcgcccttgctttgggctggcctgttagtaattcgtttttagtggaatcgaatacaacacttgagcctcggaggctcaggtcgtcggagcaactacaccagggccggtgcacctcggggcggcggcgcaggactaggctaggccgggccgggccgggcagggcggcgcgagtcgggggtggggcggcggcgcagcgccgtcgtcgtagtccccgcctcttccccaggggccgcgtcggagcctcggcggcggcggcggtgcttacagcctgagaagagcgtctcgcccgggagcggcggcggccatcgagacccacccaaggcgccgtccccctggacttccagcgctaccaagcagcaacgtccgcgccccttcagctagctcgctcgctcgctctgattccctgctgtcgactgcgccatggcgttggcgttggcggcgctggcggcggtcgagccggcctgcggcagccggtaccagcaggtaagcggcgcccgactccagccccgaactccggtccctggctctgccctgcccgctggccgcctcaggcctctctggccggcccgcggccaactcgacgccggggcttggcaggccgcgctgggcctggaggggcgggtcgggccccgggctcggattcgaggctgccacgggcccggcctgcccgtctgcgaccccaggtgggactccggcggtggaaagtcctcctcccggcctcgtttcccgcatttcccagggcgggcgggtccctgagtcagaacccgcgacccacaggcggcatcgatctgaggcattcgtttttcatttttccacctttggtttgatgtcattggaaaagaaagaaagaaaaaagcatgaaacggaaactgggtggagagaaaagattaaaatctctgctgctgccgatagctccccacagcaagatgctattgctcagggtttcagagcctagtttgtcgaaacccatccccctcgcctccgttttaagacaaaagtaaaaaatgaagcaagagacaagcccgcagtttgcagtagatttggaagaggcgatgtttggctgtggtgatgccccaaaagggagagctacgatgaagtctgcagggcgtgtgccgggtgtattttaaatgcttggaagtgacgttttaagagatttgatggcatagctggggaggaaatgtataggaaatgattggatcattttctcgggtttgatttaatacattgaggccccagggaaaagagctgcttgtttcactccgcgtctgtagctaagttcccccaggaaatttagagaccttctgcagggtttttggaagtgttaatctaacatacagtaatggtgggaattgagccaggtggtaaactgatgttgattggaaccgcaaataacggcttgtgtttgtcatgtttcagatgctttcggtgcatacagatgtcaatcctccttttatcaaataaaatgtcctatcttagacgatctacaattttatattttagaagtaaggaaagccagccagtacgatgaaaacgtagattaaccctggtgcggccggccggttactggtttgttttgctatcatcagccatgtgttgcaatccgtccccactcaggcttggactcccaaaatgactagttaagaatttcacttcttggttttataaacacggcccaaattgtggcaatgcagttaatccttagatctcttcagagcacatggatctagcacatgctttttttctatgaccttgggcagcctgcgggttccttactgctttaataaaatgggaataacagatgccacttaactgtcaccacagtgtttgcaaatcacccctactgtatactgtcatctgatttcatccttttcatcctgacttggataaggctaacgagcaaaagagctgtggtttttctttgtactgccatcttcttgagctattaggtaatgctgacatactgctagaatataaaaaatttgctggagtatcaaaatcatgtattccttagtgtggcatttaatattttttaattatgcagaattgaagtataaaaaaaagagaaaaaaatcttcctagaaaaattgtaaaagatgaaagcccaaaaaactgcaaaaccctccctaaacaaaaaccaaatagataaccaaaggtaatattttaatgtgcattcttgtgggtttatagctatgtaaatatacatagttatagatcagtatatgtgtgtacagataaggaatgcaggaaagctaatattgatttttttatgcaacaatcacagtattaagtaatttacatccattttttttttcttttaattcataccaaacccttatgaaccagggtttgttctcattgtcaaagactcaaagtggctcagtaacttgcctcaaggtcacaggctgctaagtagtgattataaggatatacacacactcacctacttaaaacacaggctcacagtccagatataggtgtatgcattagcagatatatacacatgcatatgtattctcttatgtacccatatatacgtatacagacatatctttgtacatttgtgtgcagttttatgagccccgatcatacttgcacatacttttgcacgtgtccatgtgtatcttgttctcagacatgtgcattcatacatttgcacatgtgtatcatgcacatgtatctacaaaggcatgctagataaataatgaaataggttcataattctattttgtaatgttcttcacacaatgttattaatgtcttctatataaaatatttttaatgtctgcatggcatgtaattgtatggctgaatcataatttaactaattgcttatctttggtcactaagaatgtttacagtttttttcattgaaatttatttcacatacgtcttcaaccgtttccttagcataaattactagccatggaaatgcctggtcagcgaatatgcatttttaaaaggcttttgaattatagaatgatggtgaattgtccaggcaacttgataggagcatgatactctttggttgtcacacagggctgttctgacttctatttgatctggttaatcaaattgagctagttttctcaccaagtaaatgtaatggaaaactgtatgattcattcataaatgcaaacacagttttgtctttatggtctttttattggtagtgtaatatgtggacaacttgaatttagcatagtgacagactatgcagaaagggtaggctcaaccaggtgtggtggctcacacctgtaatcacagcactttgggaggccgaggtgggcggatcacttgaggtcaggagttcgagaccagcctggccaacatggcaaaaccttgcctctactaaaaatacaaaaattagccaggtgtggtggcactcacctgtaatcccagctacttgggaggctgaggcaggagaattgcttgaacctgggaggcagaagttgcagtgagccaagattgtgccactgaaccccagcctgggtgacagagtgggactgtctcaaaaaaaaccaaaacaaaacaaaacaaaaaatggccaggcaaggtggctcatgcctgtaatcccagcactttgggaggccaaggcaggcagatcacaaagatcacgaggtcaagagattgagaccatcctggccaacatgtgaaaccccatctctattaaaaatacaaaaattagctgggcgtggtggcgtgcacctgtagtcccagctactcaggaggctgaggcaggagaatcgcttgaacctgggaggcagatgtttcagtgaactgacatcgtgccactgcactcctgcctggcgacagagcaagactctgtttaaaaaaaaaagcacggtgtggtggtgcacatctgtaactccagctacttgggaggctgaggcaggaaaatcacttgaacccaggaggcagagtttgcagtaggctgggatcgcgccactgtactccaggttgggtgagcaagactgtcttaaaaaaaaaaaaaaaaaaaggcaccgaggtgggcggatcacaaggtcaggagatcgagaccatcctggctaacatggtgaaaccccatctctactaaaaatacaaaaaattagttgggcgtggtggcacgtgcctgtagtagtcccagctactcgggaggctgaggcaggagaaacccgggaggtgaagattgcagtgagccgagatcgcgccactgcactccaggctgggcgacgaagtgagactctgtccaaaaaaaaaaaaaagaggctaggcttataggttcccctggcattattttattttttttttcatttttgccttttgaaactgagactgtctcagacagtatggaagcactattttagaaagacctaagtaggcccatactgagtgctgagaaacctgttgcgtgtttctctctctgaggacgttaactcccgttcatccatagatgaacgcagatcttctgcggtagatgtgtgcatgaattttgccaattcatttcaagtattttgggatacttggatttgaattcaatgtaagtgggtgatgatgacactgccatgtaggttcaaacagattacagaatgtttagttctggacttgcctcattacctgctaaagaagttgcccacttgtgagcatcaccaatactctgtatgatagtttgatgacattatagtgcagattttggaatggtatacttttggataaattctctcaggattatggaggaatattttgatgagcataagaatttcatctctgataggtcaaaccatattcatgttaaattattagatgaagtaatgatcctggaggaaggaatgaatagtgcttatgtggcttttttgcttatttttcagtttcaaaagaagttatatcacagaatgccaacataacctaaatgtcaaagtttcttaagttatatgctgtatttgtggcctacatggtcagtgatcaaagaaaatttataaaaatattaggaagatgggtaaattattcctttgaaaactttaattgtggtaaaatacacataaaatttaccatgttaaccacttttaagtatacagtttagtggcattaagtacattcacattgttgtggaaccagtccatctccagaactttgtcaccttgcagaactgaaactccatatccattaaacaataactctccattccccctcctccagcccctggaagccaccattctactttctatgtatttgactaccgtaggtacctctcataagtggaattagacaggatttgtccttttgtgactggcatatgtcacttagcataatgtcttcagagttcatccatgttgtagcatgtgtcagaatttcctgcctctttaaggctgaataatagtccattttatatatgtaccacattttgtttatgcattcatttgttgacggatgcttggtttgcttctaccttttggtcattgtgagtaatgctgctataaaaaacatgagtctacaaatatctttcaagtccctgctttcagttcttttggatatacccagaaatggaattgctgggtcatacggcaattctatttttaagttttcgagaaactcccatactgttttccatggcagcttcaccattttattttcccaccaacaatgcataaggattccaatttctccatatcctcaacatacttgttatgttctcttttttgtttttgtttttttgatagtagccatctaatgggtgtgaagtggtatctcattgcagtattgatccctatttccctaataattagcgatgttgagcatcttttcatgtgcttattggccattgatattttctttagagaaaatgtgtattcatgttctttgttcatttttgaattgagttttttgttgtcgagttgtaggagtaatttcttcatgatgtttacattttcaactttttactatggaaatttcaaacacatacaataataaagtagtgtaagaaattttatgtaccatcacccagcttcagcaattgtgaatgaatggccaatattatttcacttatactcaccacattattttgaaacgaatcctagaaatcatatattgataaatatttctatgtgtaacgctaaaagaaaacaaatggtatgatatttcaagaagatatcacatgaggaatatatcatgagagctctcttgtaatctgtgaggagtatggaaacaggatttgtcagtgtcgggctgtggatccataactaactgtcctttgtattgattgggctcactcttgccaagcctcatgctttgtttttgtagcagtgatagattcctgttaatctatccttgttttcttacctgatcccatgggtgaccacttgttaggcaagatttatttggcttaatcatactatctgtgtcctgaaaatagggcagtgcttcctggtttgggcagaaaccaaggcccagttgaactgctctagggaattttgctctaagctgatgccaccagtagtttaactgcacataacttcaaattgttttatcctagacattaattatatttcataattttatggtagattataaagtaaatcctaccatgagaataacttcatatttttattaagaaagaaaaaaagtctccaagagattcaagtgacatcctcagatttatttgtttttagggctttaaaataattattttggtttcttaaaatgttcttgttttacctttaagcaagtacaacagcttagaagaattatattcttttttgtttgtttgttttgagatggagtgtctttcttcttgctcaggctggaaggcaatggcgcgatctcggctcactgcaacctccgcctcccaggttcaagcgattctcctgcctcagcctcctgagtagctgggattacaggtgcccaccaccacgcccagctaattttttgtatttttcgtagagatggggtttcaccatgttggccaggctggtcttgaactcctgacctcaagtgatctgcccgcctcggtctcccaaagtgctgggattacaggtgtgagccactgcgcccggcctatattcttataataatggacaatttagataaagcaactttaatacttctgttccattcatactgaaaattggcatgatggtttatctttttttcaagtcattgttgccaataagctaggcaactggcaaacatcatgtatgaaattttttgggcttgacatactttccagtggtaattgcttctagtggttggctaggtgggggtatgagttagagtaggctctgggtatccacaggttctacattcgcagattcaaccaactgcagattgaaaataggggaaaataaccatacaataataaataatagtgcaagtaaaatatacactataacagctattttacatagcatttacattgtattaagtgttataagtaaagatgatttaaagtacatgaggtaggttatgtgtaaatactataccattttatgtcagaggcttgtgcatcctcccattttggtatctgtggggagtcctggaaccaattcccagtggatactgagggatgactgtatgatatgtcttgttaacttgaccaagagtggtagtaaagcaagtgtaacttgatatcacttagcacatcctgctgttacaataaggcttactcatgatttttctgaataacataatttatattaaatgtcctaaatactagcactgatcaagtttaagagcattcctagtgaaatattttcttattaatgcagttcccttcatatgtatatttatttatttatttccttccttccttccttccttccttctttctctctctctctctctctctttctttctttctttcaacagagtcttgctctgttccccaggctggagtgcagtggcgcaatcttggcacactgcaacccctgccttccagtttgaagccattctcctgcctcagcctctggagtagctgggactacaggtgcccgccagcaggcccggctaatttttgtatttttagtagaggtggggtttcaccatgttggccaggctagtctcgaactcctgacttcaaatgatctacccaccttggcttacagagattacaggtgtgagccaccatgcccaggctaccttcatatgtatttttcttttctcttctctcctcttcttttttatttatttatttatttatttttgagacagggtctggttctgtcacctaggctgtagtgcagtggtgagagctctgctcactgcaatctccgcctcctgggttcaagagattctcgtgcctcagcttcccaagtagctggtgttacaggtgcatgccaccacgcctggctaatttttgtatttttagtagagatggggtttcaccacgttgcccaggctggtctcgaactcctggccttaagtgatctgcccatctctgcctcccaaagtgctgggattacaggcatgagccaccgtgcccggcccatatgtataatatatttcttatccccctccccgagcaatttagcataatctttgaccatttttctgtttttgtcttttaaattagtttatctcagcaatattaattagtacttaccataatcaaaggcttgaatacaatttggaggtctatctggagactggacaatttatgaatagagatataagggtttagggagtggtgcctgtttaataatgatttcattttctttttttgctttcttttttttttttgagacggagtctcactctgtcgccaggctggagtgcagtggcgaaatctcagctcactgcaacctccgcctcctgggctcaagtgattctcctgcctcagtctcctgagtagctgggactacaggcgcccgctatcacgcccagctaattttttctatttttagcagagggggtttcaccatgttggccaagatggtctcgatctcttgacctcgtgatccacccgcctccatctcccaaagtgctgggattacaggtgtgagccactgcgcctggccgatttcattttatcattcgatgcttaaatgtgatatttaaaattaagcattttgtattaaatagtaatacattcttcatgtgctagattcaaacagtaactttctaaagaaaaacttgatttccctttcctccctatctctttgcagagctgtaactgctcctcaagtttggtgtgtgttctacaggcttttttttttttttttttgagacagggcctcactctgcacccaggctggagtgcagccacatagtctcggctcactgcaaccctccatcttcaggctttcttcttaacctttccttggctttaaatgttttttaaataaatctgtactttcaaatagactttactttttggaacttttttccccctaagatgggttcctgctatgttgctacccaagctgggcgtcaaactccttggctcaagcgatcattcccccactgacctcccaagtagctgggaccacaggcacacgccagcacacccagctgaaacatttttaatttttcagaaacactgagaagacagtagagagtttccatatgccctatgcccagctttccctattagcatcttatatcagtatggtacggttttcacaattattgaaccaatattagtacattgttatcaattaaagtctgtactttgttcagattttctaagtttttacctaatgtcctttttctgttccaggatcccatccaggatccaggatgatataatgtggtaatgttacatttagtcgtcctgtctccttaggctcttcttggctgtgagtttctcaaactttccttgtttttgatgagcttgacagttttgaggagtattggtcaggtctttgtagaatgtccctcaattggggtttgtttgatgtttttctcatgattagactggggctgtggatttgggggaagaccacaggtaaagtgctgttctcatcacatcatatcaagaatgcacactttcaacatgacttatcattgttgacattgaccttgatcacctggctgaggtagtgtttctcaggtttctcttctgtaaagatacgttttaccccactttccatacttactcttgggaaggaagtcactatgcatggtccacacataaggagtggggagttatgtaacacctcgctgagggccaagtattgacatgaattatttggaatttcctgcatggggcatttgtctctgctctgataatttgtttagtatttatatctgtatagactcataaatatttattttattcttccaattatccaataactgctttattttattgcttaaattgcatacctccatcattgtggatttttttttaaccacttccatactttttggcactaggctcatcttatatatttcctgcactatcctagaatcaggcatttttccaaggagcctaggttccttttattgaaaaataatactacaaaccaagatctgtgctccttgctactagggtgtcattgcttttaggccttctaagctgacagagcaaggaactatatatgtctgtatattaacctgtgggtatacacatctgtaactatttctgtatgtaccatctatctatattaagctaaacataagttcatactaatatctccacctctaatttgttaccatatgggtcactctagcctactccccttatctataaactcccactccaacatggagaaacctggctcacttccaccatccatttacatcattcttcagttctgatatacatgtgtaagttgtatcagaattattaacccacacccctgtgggaaatagtattataaccactacaggacaaagcatatgtacagttcctttgcctttagtcttaaaaactctacttatttccaaatgttacttaggtcagcaccttttcctccacccacttgagtgaggttgtttcaaacatttataatacagttatcttgttttgtcacattttgcatacatcctccagccacctaagtggatttttaaaatttgcataccttgagattctttctttgtgctatcaagttctataaattttcacaaatgcatagtgtctttttttttctttttaaacaacacgttttaagaaaaatggtaggactgatttttctttattttccaaactttctgctaatgatcatgtttgctttctttttaaagtacctttaaagaaaattttatttagaaataattttaaacttagagaaggttgcaagaatagtataacataacactcatatatctattaccaaattcatcttagacttattgttaaattttgattaacaatgaatacaatcctataaaaattgaaagcgctacagataaaacccaagtccctttaatcatcacagtcctttctctatagaattcagtgttgatttagctacttgttgtcaaattattattattattatttttgagacggagtctcgctctgttgtccaggctggagtgcagtggtgccatctcagctcactgcaatctctgcctcccgggttgaagcaattctcctgcctcagcctcccgagtagttgggattacaggcacgcaccacaatgtccagctaattcttgtatttttagtagagacggggtttcactatgttggtcaggctggtctcaaactcctgacctcaggcaatctgcccacctcagcctcccaaagtgctgggattacaggcatgagccactgcacccggcccaaactttctttctgtttataaatctcgtctacagccataccactctgaacatgcccaatctcgtctgtttataaatctcattttcacttgtacttctaccagtacctggtgccaccagttcttgagcttttgggggatacaaaacaggttgttttttggctttctggtttaggatttagctttcttgggtctgctgagtcagttaccacttgtcatcctgcttctagctttcagaaatttattgctgcttcctttcctgttgcttgtaagtttttttgcctttaaacaaacaacattttactcacatttatatttaagagggtttcaggaggaataagaggcaactgtctaatctttaattggacattatacagtaagtgtaattctttgtgtttcatttgctaaacttgcagacttagtgaggccaaaaacaccagccatgtcactgttctgtccccagaccccacatcagttgtggcctgacacatttaatgagagttgttaaattaacctagcagttgaatacttgtagaatgaaacattctgtgatgatagtgtaacaggaaaggtattaaatgcttgatgcacctactttttggctctctttgttcttgaaggaagaggggacctcatgaccaggcctgaatcaaggtggactctcctgaatagaataccttttcccacccataatgtctacctgagtatgctctactccaggctaatgagcaagcatagcttttggaacttctcactatcagagctgagcaggctgcagtcaacatgcaagcctgggttctaggcatggctctgtcactgtctttcagtgtctacatttgatttcactgtcttttcaatgtggaatattccattcttgggatatactaattggttgtttttagaagaaacaatattgtaggaattacattgtgatcaagataagttttccttcatatataatacaaactttaattgaacacctgagcacttaatgctaagtgctgtaggttgaatactaaatgttaaacatttctcatttctaaagagataaaggttggagatgcctgtttctcctttgtgtttcttttctgttatttctttctaaaattggtaactataaagccatactattcttttcatttttattgtgaaattatggatacatatgaaaggaatacatagtatagatataccttgtgcaaaataacaataataaatcagagacctgtgactgccatccaaatcatagcaatcttttttgaaagtttgtttgtttcacacgtattcttttagaccagtcagtaggagagagctgcttttgccttagttgctttgtaaggccatgtttctcctcaactcttccatgtttaaaatgcactccttgtgctgagtactgatggggataccatacctgctgttctcctctcctgaaaccatcatcactgtcctctctacagttatagcagtcatctgtgtatggctacatgcatagcctgtgaatttatttggagatttgtaaagtaatatgaatacttttcatagatgcgaactggtaagttaaaatggtattaagattagctgctatttcttcagctaagtttctgttagaaatccacctccatgcctgttttggccaacactcactaatgtatccatgctgtgccactcactggtaagtgctggggtgaacgcagtgataaaccatgcaaaggtctcagtccacagagggctcacagtctgcatcactattcctcgtacagtctttttcttctcccatttctgaattgttagtgaagtatgatttatgtataaaaacatgtacaatcacaaatgtgcagctcaatgaattttcacaaactgaacatgcacagctaactagtacttaaatccagaaacagcattgccagcactccaaaagatcccttcagtgcctttccagtcactctcctcccgggacatactcactggtatgtttagtttgcctgttctttaaaaaaatctttatatgaatctaatcatacagtaaatactgttttgagtctggcttcttttgctcaacatgatgttgcaaaatttacccatgtgggttacagtttattcatgcacactgctgtctcattatgtgaaaataccacaatttatttacctattctactgtagataggcatttgaataatttccagttttatgaatactgttgctttttccctccttatgtgtcttttggtatcatatgtaaatgtttctgttggacatataactaggagtggaattgctggatatgtgtaaattcattttttgttgatattagcaaacagttttctaaagtagttgtacaggtttatactcccatcagtagtgtagtgcgagaattctggttattatatattctcaacaacatttggtattttctgttttttattttggtcatcatctgtggttttaattttcatttctataatgactaatgaaatagagcacctttttgtaagtttattggccatttagatatctttttttgtgaaatgtctattcaagtcttgtccctttttttattgaatggcttccttattgattttaagaagttctttatatattttggatataagtccattgtctgaaaaatatatcctcccgctctttggattttcttttcactttcctaatggtgtcttctgaatgaatagtagagttctcgtgtgtgtgttttggttttgttttgtgttttaggcagagtctcgctctgttgcccaggctggagtgcagtggggtaatctcggctcactgcaacctctacctcccaggttgcagtgattctcgtgcctcagcctcccaagtagctgggactacaggggcatgccaccacacccagctaatttttctatttttagtagagatggggtttcaccatgttggccaggctggtctcaaactcctgatctcaagtgatcgtcccacctcggcctcccaaagtgctggtattacaggcatgagccactgtgcctggcccaaacaagtgatttttaaaattgttcttgaaatctaaaagaattattttttttaagaggcagggtcttgctatgttgcccaggctggtcttgaactcctgggctcaagcagtccttccacctcagcctcccaaagtgctgggattacaggtgagatccaccatgtccagccataaaataattcttgaatagatgccagttgtagtccatggtcttttgtgacttttttttctcctttggaaggagctgcttaatgtatcattttaaatgacaaataattaggagcttgtttgtgcttccatgtttctattttagagctttaacatatcttagatgaaatctatatacggggtcttgagaggttaaaacagaatgtatttaccacctttgcatttaaattatggactagattttatataaattattttaaaatgagtctgatacagccattatttttagtattgtttactctgtaaacaaaagatgaccagttcctgctattctgtcaaagtcagggagccaggtgtgtagtctccttttcaacttgtctttctctagggctttaaatccttctcaaatgggccctggagctctgagctaagcctgaattttcttatggaaccaagaagtggagaaaaagaactcacattactgaggatatggactggttataaatagtgtagatgctacttcttaatttaaaatgctatatgttcatgagcagctagacttgctattaactatttggaaatattgaacaattgtcgctccttatttccgtagtctttgtaagatcatgacccatcaaggacctaccgttaacctcaaaggctcttggctgtaggttttcctcaagctgggtgccagaaaacagaggattatttgcttgataatatcttggtggagccatcagtactagaaatatttggatgtcagacacagatcccaggggttggcaggttgatgataattttctggtgccaccccaaaaccttgcagtgctactaccataaccatggttggctaattagtgccttcggggatgagggttgatttctgctagttccttgcctttctctttggatggcatgccttcacttgttacttggacctatgtaaagaataggaagatcagaagaaaaccataaaattgatgatcataggtcacagtatttgtgctttcatttctaaggactcattctaacataaataattttctctttcaaaaccaaggtgtactcatcagttatattgatacatattaacagtgatgataaccacaaaaattttaaatagaaagctttacaaacggttagctttgtgaggggatttactgcaaaattagggtattcttttcatctgttattgtattgactttttaaaacattattctgattacttagtttttcctttggcaaattttgatttttaaatgtgttttcattttgctattgagaggattttcatttatcttttccatgggagatattgaggatagtctaactctggtaattggtttggtgggtttttcttttcttttttttggctccctatgagcaacttgaagatttttttttttttgagacggagtctcgcctctgtcgcccaggcgggagtgcagtggcgcaatctcggctcactgcaagctccacctcccgggttcacaccattctcctgcctcagcctcccaagtagctgggactacaggtgcccaccatcacgcccagctaatttttttgtatttttagtagagatggggtttcaccttgttagccaggatggtctcgatctcctaacctcgtgatctgcctgcctcggcctcccaaagtgctgggattacccacacccggcctcttgtactgtaatgtttttaaggttcatccatatttcagcatgtgtcagtatctcattttttctgggtgaataatatttcattgtacagataagccacattttgtcgatcccttcgtaagttgatgaacattgttggctattatggataatgctactgtgaagattcatgtaccagtttttgtgtgaacttgttttcagttctcttgggtatataccttggagtggaattgcttgatgttaatataaatctgtgttgctgtttttgtttgagacaggatctcactctgttgctcagactgagcaacagactagactggactcactgcagccttgaactccttggctaaagtgatcctcctgtctcagcgtcttgagtagctggatcacaggtgtgccgccatgcctggctaatttttaaatttttagtagagacaggatcttgctgtgttgcccaggctggtctcagactcctggcctcaagtgatcctcctgcctcagcctcccaaagtgctgggattgcaggctgagccactgtgttttcagcctatgtttaacttattttgaggaactgccaaacttttccacagcagctgtaccattttacattcccaccagcagtgtattagagttcccgtttctccacatactcagcacttgttattgtctgtcttttttattatagccatcctagtaagcatgaagtggtattgagtcttggttttgatttgcgttttcctgctgacttaatgatgttgagcattttgtgtctactggccttttgtatatcttctttggagaaatgtctgttcaaatcatttgcccattttataattgggttgcctttttattgtttagtctctcttatcttgtcagaattatttaaaatatcttcccctaattaataacaattagatagagaatttttcttgaatgtaaataatttggtgatgttagaataacagagtttatatttacaatatataaagtaaatgtacaggccacacaaacttaatgattatttctgaacagcagtaaaatagcatactcagctgtttaccaaaagaaatgtgcttgtttactttttgtaattatactgccttgttttggtaggatttagggactatgcagaagtagtgggttggtaatgagagctttaaaaattgcattttaaggtaaaagttttagcaattaactctaagatagaagtagtaaataacattcaaaactagagttcaagcattgacccagaattttctgatatttatttttaataacttaaagaagggacttgaaacttacaagaattttcttgctgtttaaatatgaccaaaccaagaaaaaattaagtagtgaaaatacttgcaactttatggacttactggatacttcagcattatttttcacgtgctttgtttgtttgtttgtatgaattctgaggtgtatgttgataccttttctcatatataaggatttatgggttttgtttagtgtagggttttttgttgttctctcattcttagtaaatatagataatccaagaatgacttttaagtgcctttaggtcctgccatttgctacagttataaatgttgccccttgcttagccctaatcctagccccagctctgctattgcttaaaaacaactcattttagaactgcttagtgaggagggcggtggtgaagctttcttgctgattgggaactcaaataatagtttaaatctgtagggtataatgtcttaaacgtgttaaattcaagtttgtttctattagaattagaactatatatgatttctgtgttggagattcataaaaatgtcatatttatttttaataacaataggacctagatacagaggaaggtgataaggagggaaaaaaaaacgctctattttattttatttatttatttatttttgagacagaatctcactttgtcgcccaggctgaagtgcagtggcttgatctcagcttactgtaacctccacctccccggatcaagtgattctcgtgcctaagcctcccgagtagttgggattacaggtgcatgccaccacacctggttgatttttgtatttttagtagagacgagattttaccatgttggccaggctggtctcaaactcctgacctcaagtgatccgcccacctcagcctcccaaagtgctgggattacaggtgtgacttgagaacaaaaccttagtagcacctatttgtaaataaatgttttcttaaaattgaaggtcatcaaggccaaaggcaccatggcagattgtgacatatctgaagtaggaagagttttagggttcttctggggagccattctagtctgctcagcacatagaagggagcaaaggtaatcaggaaaagagtaatggcacactctgggtgtgagaaagtgaaaggtcatctgaattctcaggtgattggattatttatttcttagcgatggaggcataggtagttataggtagttctgtattaaactctactttgtatggcctgtgtaggtaattgtatagctgtatttggaaaaaatgccatttaaatcatatttcagccgggtatggtggctcacgcctataatcccagcgctttgggaggctgaggcaggcggatcacttgaggtcaggagtttggtaccaacctggccaacatggcacaaccccatctctactaaaaatacaaaaattagccaggtatggtggtgcatgcctgtaatcccaactacttgggaggctgaggcggaagaatcacttgaacccaggaggcagaggttgcagtgagccaagatggtaccactgcactccagcctgggtgacagagtgagattctgtctcaaaaaaataaatcatatttgaagtttagtaaaattggtcagaaattatatgacaggaaaaaaaatccagtgtccaacagctatttattatctatcacagtcactcaaaggatatcccccctacccttgcccaacatctctgtgcactgagagcagaagttcccagatctgtcccagttttcagatctgtctctctgagtatcccctacaacccagattctgtgacagttaaattggactcgggcttccactgacccagttgatggaagagactgagcatctttccaactctaacccaaacgtgacaatgccgcgtattctttcagaaagtgtcaggaaagagcccgaagatccctgagctccagggacatggcagggaggctgcacagaagggttcccaggggtggaaaaggggaatcttaaaaataaattttggaaccaagtaatcaattttatagcacactctagttctagcgcacatggttcctccacttgttagtgaatgcctgacttgttgcccctgaagtgtaaaaagatgtatatttaaggattccattgttcagcaagttattatgcatgcagatggcaggcactaagtgcttactctttttgaaggtgattgcctctccccgcgatccccctacattgcagtgttttgttttaaacagctttcttgatggagcctaatcttatcaccagcattatagaaataagccagagtgagaagcctgtacagtgaggtcttgggattataccatagtcataatggtgttttaaattgtttttatttttcaattgaaaaaagtacactattctaaagtagtgaattttagtggagcaaactcttcacctggaagtgtttttttccttaccaaagtgaaaattggtggttctaagaaggctggaatttatttgcactgtcatcttttactatggtgggggatgatcaaatttaacatttccaagtttccaggagacaggcagcctatcttataaatattgtcatagaagacctcttaacctctttgttctttggatttctgtatttcttagaattgtcatgcttcctttctgagacctaaattatgcaggggtaacgtgattactttgacaccaagctcaggaaatataaaacgggttaagtataaaccaagctaggtttaaatgaagttcattactggctacagtatattaagggaacatattcgaaaagtctgtatttgcctttgttcttgctgctgactcttctgcttcttttggtgaccttcccacagcacaagagtggttaggtgtgaggaggctaagaataccttgccaagagcttcaaataatgttaaaaccagtattttaggtgaaccatggtcccaagaactctatttaagtaattcctagttcggtgtcccaggagcatttggctttactgcacctattttggccacatgtctttgtttaaaaggaaaaattatcttaggttataatacagctattttgggagtaccttaagtttaacattagattactaggattcgaaaaaatgtacccagatggtagatgctgatgagaatttgacacaagaattcatttgaaattgaagcttttgtagtattagtcagaaaaatttattgacatctggttttgaacagcatagtgagtggagattgagatgatcagttaaaatttaatggattgtggccgggcacaatggctcacgcctgtaattccagcactttgggaggctaaggcaggcatatcacctgaggtcgggagtttgagacgagcctggcccacgtctactaaaaatacaaaaattagccaggtctggtggcatacgcctgtagtcccagctacttggagtctgaggcaacagaattgcttgaacctgggaggtggaggtttcagtgagccgagatcccaccattgctctccagcctgggcaacaaaagcgaaactacatctcaaaaaaaaaaaaaaaaaaagaaagaaagaaaatttaatggattatggagctctacttcacattagtacacctcggtctctctgtttttctgtctcaattcaagatagagttctttgtagcggtatggtttagaggtggtatgtattttacaagtatgtcagttggctctgtatatcctgatgataccgaagccaggaccagacccctacgtcagttatatcactaaatattctgttattaattttcagattggctcctagtcccagccaactcatgttttaaattaatatagttcattgtcacactggactggaaaaaccaaatgaaatgtctttattctatgaagatgacttgtgtagactatacagactagcaacagcagacactttcagtgcgagttattttaaaaaattgaagatttgactggcagaaaagttgctgagatagggaaaagggatatctattgacagatatatgtagaattagggagaattttatgttttcataagtgtctcaattatttaagatttaatttggtgtgcatgtggaatataatggtaacatggtttgtctgtgatttgtcttttgtcttaacagattatttttctaactttaaaaaagtatacatcacttgaggccaggagttttaaaccagtctgggcaacacaggaagaccccatctctacaaaacataaattttaaaaaaagttagccagggtcacagtgtgccataattgtgccactgcactcctgcctggacaacaagagtgagatcctgtctctccaaaaaaaaaaaagtatgcaagtgataaatattcactacaaaatatttgaacatgcagatacacaacaaaaaggaaaaattcccctgtactcctattacctagaaataacagtgttgaactgtaaatgtctcattctaggcctcctttccattatatgtctgcctttttaaaaaggattatatgatgcctattgtttggcaagtttagcttttttcatgtaataatagtacactgtaaatatctatttgagtaatgaaatttaatttttttttttttaaagacatggtctctctcatgccccaggctggagggcagtggcacaatcttagctcgctgcagccacgacctcctgggttcaagccatcctgctgcctcagcctcctgagtagctagaaccacaggtgcatgccaccactcccgggtaatttttgtattttttgtagagacagggtttgttttgccatgttgcccaggctagtcttgaactcctaaactctcctaatctcaagcgatcccacccacctaggcctcccagagtgctaggattacaggtgtgagccaccatgcctggctgaaatttaatgtatattttttttgacacagagtttcactctgttgcccaggctggagtgcagtggcaccatctcagctcactgcaacctccgcctcccgggttaaaacgattcttgtgcctcagcctcctgagcagctgggactataggagtgtgccaccatgcctggctaatttttgtatttttagtagagattgggttttgccatgttggccaggatggcaaaatttaatttttaatggccgtttagtagtcctttgtgtgatgtactataatttatttgtctcctatgattggaatttaggttgtttctaatttttgctgacacaacacttatatgtacccattttgcctctttgaatatttccatagaataacttcctgggagtagaatacttttcttaaaggatttgtgcatttcaaggctttcaaaatacgttgccaaatagctttcgagaaaggctatttatttcagtttttgggttcttatctttctaattggctttatgcttacattaaaatatgaaatttttatttagttgcagaatgaagaagagtctggagaacctgaacaggctgcaggtgatgctcctccaccttacagcagcatttctgcagagagcgcaggtaggtaacagggcaaggtgatcacggaatccttaaaaacactctgtgctttgacattacagtttaaaaaaaaaaaagcttcagaagcagttggaagaaaggttacaaagcaagtctggattttaaataattgagaagtggatacatttaaaaatcacccccagtctcttacattcatctaagaatgtcttttggttcaattgttatgtatgtactcttaagggaatggctgggttttgtctgctagagttagtggaactccttttatttcaattcagctaatcatcaaaaaattgtgactgttaaaattctgcagcatattttgactacaaggatgagtctgggtttccaaagcccccatcttacaatgtagctacaacactgcccagttatgatgaagcggagaggaccaaggctgaagctactatccctatggttcctgggagagtgagtataatttgccatgttacttgatggctgctctgtgggaattaagttattttcattatccaattttgattcgttacttatgattgtattgattagtgaaagtagagctaattttaaaaatcaggtacatttgcttttgggataataaactcatctcttcttaaaaatgatgaggtaaattactagtcagacctgctcatttttccttcatctctcctattaccactactttttcaagtctcctgggatggctgaagtatcttgaagcagaagcacactaatgtttccttgattctgcacacttaatattttgctgttgggtggatagctgctagggaggtctaggaagagaataaatatatgcaaatttgtagaattaaactcataagagggatcttcacattgatttagatgaactccgttcttccagcagtccagatcatgtcatttgtggggacatagtgagttcagattccatctagcctgatgttgattcatggaggtggagattgggatgccatataaaccagtggagattattgggcactcttggttcttgatgtcagatatgtgagtgctgcatttgtcattttgtaaatattcatccagattgaggctgatgaaggtagggcattgtgggagaatggaagcaggataagtccccagaaatgcggggaccatcaggcctctaggctctattctagcaagtaactggagtgagtgatgagcaagtacagaagcctagtctgggcagtgtgatggcataggcccagatgttcccctcttggctttaagatagtagacagtttgcagaccacacctactgtgaggagcgaagctcctaaaatcattgcttggtagaaatagtttatgagcccaatctaggatgatcctaaatggcgatgatgtgcattcagttcttacaggtaccagggagaaactaaaaaccctagtgtctaagggcaagaccatcataatgtaactgccgtatattggaagcaaaccacatgcttggtactgtgcttgatgcttttatatattatgtatattgtttccaatcttcatagtaaccttgaaaggtagagattatattatctccattttacagatgtgaaaatttaggtccagagagaaaaagtaattttcccaaagtcatatgactgataagtagcagaagtgaaagtcaaacccagatattctagaatccaaagtccttgtttgtttcactgtactctgcagatctgttgcaatatatggtaaatagatcagtatggaaattagctctgattaatcacataactcttcagaatactttcatttgatacaagtacatatttaggaacttcttaaagggctctagtattcagcagacatgtataaagcactttttaaatataagatgcagtatattttacagtggataggtaaaaagctttgtataaggcagtgttcctcattgttattcaaaagcctaaaatccaactagggtaaagggcccacaaatactgataactaagacacataagagcagtgggcatcaagaactgaaactttttactgggtgatcataggggaaaatgagatttgatgtgggcccggagttttgatgggtaagaacattccagagggaaaaaggcacagaaagcaggtgtgaattcagccaggtgaaggcagttctgtggatgtagtaattggctttgtatgagcaagagtttgtgaagggagatagaaagtcctgactacaactgttgtttatagaatctttggaatgttagatgggcgtatttaaacttcatatgtagcttttagagaatccttcagagtttttgagcaggggtatgagaggacttgtgtggtgttttggaaacattaacttggcagcaatttctaggatgcatcatggtagaagagataccaagaggacaggaagaacagttagaaggtgttcgtaatattattagatgttacttccatatattttttggagtatatctttgaatagttatgcaaaaaacaattagcttttaattttggcaaaaccttattctgctgatacgaagtaagaatactaggtatttaatgttttaaaaaattaattataaaaattcgtttttatagaaatttcaaacaatagaggaagtatgttaagttagaaaaggaagctgccattgccaccgacttccctttctctagttcccactccccaggggaaccactgttaacaatttgggtcttccagacccttttctgtgtacaacaagttttgatattccctaaaatatttttcatttaaggagccctcattgaactgtgtacaaatctatcacttttaagtatatattcaattatttttaaatatctcaacacacacctatatcagttaaaaagatagcccacatgtgttggtattgttcatgatcatgttatttctagttttgcaatattacgttatcaagttaggatggaaaagatcttatctgctgataaggttagaagtgagatttagatctgtataccaatacaaatacatgcttatggagttgagcagtgggcagttctgtggactattcatgtatgatcgtactattcttttttcccctatatcagcctcgtaaagcacttgagctggttgattttatctttttttttttgagacggagtctcactctgttgcccaggctggagtgcagtggtgggatcttggctcactgcaagctctgcctcccgggttcatgccattctcctgccttagcctcccgagtagctgggactacaggtgcccgccaccactcccggctaatttttttgtatttttagtagagacgggtttcaccatgttagccaggatggtctcgatctcctgacctcgtgatctgcctgccttggcctcccaaagtgctgggattacaggcatgagccactgcacctggctgattttatctctaattactaatattttaggtcctaaacaggtcctcgattactgatattctagaatttaaaccaaatgtgtaagtaaattttgtcttttgtcttttcttgaggtatatttttttaaaggaagagtaaatagttctttgtgaactattcttgaagtgtattgtttttgaagtgtattaaatttttgaagtgtattgttaaagaacttgggttataagatgaaaatttcatttagaacactggggatagaagaaagatttgcttcttttctaattatatattaataatgcaaaatggaattttaaacacacattgagcctttaatgctcataattttgaactgaaccaaagcaataacttttttccttgctactcaaatttaacccttcatttttctttatgtcttccctttgcctagaaataattctttttcttttcatttaggatgaggattttgtgggtcgggatgattttgatgatgctgaccagctgaggataggaaatgatgggattttcatgttaacttttttcagtaagtatgtatgcatatcagaaccaccccctacaaactagagtgctttggattatgggtgaatctgactgaattaaaataactagttgagctaatatattaatacattatgtttatttggtgccttttactggaatattcaaagcttaatatacctttgttttaatatcccttctctttgccttctattttcatgaatatctgtggaaaatataattaaagcagttatgaggatgctgcatttttacatgagctaagcctcatcttccaggtctacggggcccttttgcgttgcttctactcatacttaatctgtagaataggaattaaatgcctttgatgaattttgctctgaaagcttcttttttctgacagatcaaagtcagcctgattttctcccttcatgttacttgccatttatctttttgtgtgcctcacatagaagtgagcaaccatttcctttatttgttgtattagaaagataagaaaatgcaatgcttttagaggtgatgttatataagttgtcaaaattcagagaacaggaggatgagtgacttgaatcagattatttgggggcacttatttaatgttgaaggcatttattttatttactggactatatatatataacacacacagtaaaaagcagaggtcaggctgggcatggtggctcatgcctataatcccagcactttgggaggccgaggcaggcagatgccctgaggtcaggagttcgagaccagcctggccaccatggtgaaaccccatctctactaaaaatacaaaaattagctgggtgtggtggcaggcacctgtaatcccagctactcaggaggctgaggcaggagaatcgattgaacccgggaggcagaggttgccgtgaggtgagatcgcaccattgcactctagcctgggggacaagagtgagacttcgtctcaaaaaaaaaaaaaaaaaaagaggtcaaaggctgggcacagtggctcatgcctgtaatcccagcactttggaaggctgaggtgggcgaatcacctggggctagaggttcaagaccaacctggccaacatggagaaaccccatctctactaaaaacaaaaattagccgttagccgggcctggcggtgcacgcctatagtcccagccactcgggaggctgaggcaggagaatcacttgaacctgggaggcagagattgcagtgagccaaggtcgtgctactgcactccagcctgggtgacagagtgagactcagtctcaaaaaaaaaaaaaaaaaaaaaagcagaggttgtaagtatacagtttggtgaatgttaaggtttgctttttgctgttttgtgttttttttgctttttccttttgagacaagtcttgctctgttgcccaggctgaatttggagtgcagtggtgtgatcacttctcagtgtacccttgacttcctgggctcaagtgatccccccacctcagcctcccaagtagctgggacttgcaggtgtgcaccactgtgcccggcttttgtttgtttgtttgtttgttttgtttttttcagtttttgtagagatgggtctcactgtggtgctcaggctggtctcaaactgctgggctcgggatcctccaacgttggcctcccaaagtgctaggattacagatgtgagctattgtgctcagcctattaaggcgtttttgaaagtgactagcagtagacataagtgtgtaaaacctgatagggaggagtttttcttgtttgctttttggggaaagacagaagaatggatgttttagaggaggtatggtgacggttgtggaggacaggctgtcttttatcctttggaaagtcatttaaacagaggcaggtgtaaggcactcttgttcttgggtaacaggacatgtctaacatctttcccctcctctgcttttgcagtggcattcctctttaactggattgggtttttcctgtctttttgcctgaccacttcagctgcaggaaggtatggggccatttcaggatttggtctctctctaattaaatggatcctgattgtcagggtaagttgtataacagaaaagatgggctctacagagaggcaataatcagaatatttttgaatattcgatttttcatttgcttttttaaaagttatttcagtgtgttaaatgatttttggtttgtctgtggtttcatgatgaagctgaaccaaaatcatttgcaaatggttcatgcacacaagttttgttgtcaaatctattgaaaactggtctatcattaatattgttcaccatctgctttgtttgtttttaatgtttataatatgataatgtatatgttctgaaattagttgtttttataataaaaggaaggatattacccctttgatatctcatcaaaagcagccattcctctcattaattctgttacagttttaatctagctctcttcttccttcttttcttttctccttctcttcccctctcccttccaggattgcttatgccaaaatgtttggggagccaaaactgtttaacaagtattgctccattaggagcctacctctcaagtaaattacagagatgtatgtctttagtgacttcagagttaccagttagttaaatttgcatgaagtaatttacagttgtaaaagatctaggcttttaaagtatcctctgatttcagataaatttattgctatcgtcaaccttataaacaaaaccaatttatccgtcataaattggcatacttaactaactaatgggtgttgcttgaccactgtagtttttctacgtagtgccaccttatttattataaaacatttgttaacgtttttagggttccagaatcctttgggaatctgatgaaagttgtaagcttttcccccacatagctaatatgtactcatctataataatggactttaggttaagaactcgagccctaaagaggttttcattccttccctaaattttaattgttactttattaacaaagaaaatactatattttacttttcgaactacattaaactcttggacaccaaagcctccaaggaaaagagaaataaaattaattccagtcttttttttttttcaaactaaattttcaactgcatttaaaaagtgaatcaaatgggccgggtgcggtggctcacgcctgtaatcccagcactttgggaggccgaggcggacagatcacgaagtcaggagatcgagaccatcctggctaacatggtgaaaccccgtctctactaaaaaaatacaaaaaaaatagccagacatggtagcgggggcctgtagtcccagctactcgggaggctgaggaaggagaatggcgtgaacccaggaggtggagcttacagtgagccgagatcgtgccactgcactccagcctgggcgacagagcgagactccttctcaaaaaaaaaaaaaaagttaaatgaattaacttgtatcaggtaggatgcttttgactgtgagtaacaaaaacctagctcaaaatggcttagaaaaaaatttttaattgtttttttaaacggggaaattttatttcccctgaaaacaagtccctgacaaggttagttaagtcagtggttctttgacatcatcaaggacagctgacatatcagcatattggcacaggtcccccctcccctcagggttaaaagatggcagtttcacgtatcagtattacattgaaacacgacagtgctcaagagaagaaaaagaactatttaaaaaaaaattttctaccacctttagaaagaactgtttttttctttgagaagcaaggaaagcttctcagaagccctcaatttccactggtcagaatggcatcctgtgcccattcctaaaccagcaagggaaggggattaccctggtatacatgataaagcttccaaagtcaagacaaggctggaggagtaggaagaaaatcaggcctcattagaaagaaggcaatggtatcatctactcaggaggctgaggtgagaggattgcttgagcccaggagttcaaatgcagcctggacaacatagtgagatcctgtctcttaaaattaataataataataattgcttttggcactgaataggcaaagtaaaacgatcattattgaaacacatttctagaagagcaaatattatgtggggaaaatgttttctaaaaaaaccaatttttgtatactttcaaaataaggagttaataaagctaaaagctgtcatatttaacaattgcttttccttgaaaggaaatagatttgcatttattgtaaccatttcaagctagtaatatgtaaaatactttcacttatattttaaaattattctaaaggctaagtgatggcattggtagaacaaataagtcttgctggtcacggtagctcacgcctgtaatcccagcactttgggaggctgatgtgggtggatcacctgaggtagggagtttgagaccggcttgaccaacatggagaaaccccatctctactaaaaatataaagttagccaggcatggtggtatatgcctgtaatcccagctactcgggaggctgaggcaggagaatcgcttgaacctgagaggcagaggttgcagtgagccgagatcacgccattgcacttcagcctgggcaacaagagcgaaactccgtctcaaaaaaaaaaaaaaaaagaataagtcttgcattttgtgattttgtgtacccttaagttaaaatgagaaattgtgtctgttaagctgctataaagtgttttgccttattttttagttttccacctatttccctggatattttgatggtcagtactggctctggtgggtgttccttgttttaggtaagtgtttttaatgtaagaaaaggcaagaaaacattacattaaattttataagtaaaattacttgtagtaaatgttcatcttgattactgtatagtaatatattaacaattaataataaacatttttaaaatgttgctttcttaaaaatagtctttcgttaccatcagagtaacatgcacgttaataaaaagtttctttgtggttgtaggttcttcctaacctatacatatattcttctcccccatgtgataaatggggtatgctgatgactctgtagtctgtcagtttttttccccttgtgagctattgccacacagcattcacagactcaaaccagaattgcttatacgcttactgcagccagttgaactaactcataagtgcaggtggcccatctttggtgggtctgtcagtagaaaacagaaaatatgtaacctcgctagacataacttctgaaagaaccaccattggatttccagaatgctttagtttaccgttggtgagaataagaactattttacaataggcaagaggacttttttttttttttttttttttttttttacagagtcttgctctgtcgcacaggctggagtgcagtggcgtgatctcggctcactgcaagctccacctcccgggttcacaccattctcctgcctcagcctcccgagtagctgggactacaggcacctgccaccacgcccggctaatttttttgtatttttagtagagacggggttttaccgtgttggccaggatggtctcgatctcctgactttgtgatccgcccgcctcggcctcccaaagtgctgggattacaggcctgagccaccatgcctggcctggattatattttttactctaaatatttatttttaaatacttttttcctagacattaaaacaaagagatgaacattggagtaaattatttcaatgttggcttcttgattatatgtaaagaaaatatatatgatacagttagagagaccaccatcacttctgtcatttctacaaaagaataacagggccatcttgtggctattaaagatggtgtgattgcctcactgtgcttctgtgacatcagtttctcctatgtcctgttttgcttcttgttgcccccttaattaagatccttggtggtggttctgctaatggttctggctaggacatcctggcacccatgcctggcaggtgtttaataaattgaggacctgggccaggcatggtggctcacgcctgtaatcccagcattttgggagactgagtcaggaggatttcaccttgaggccaggattttaagaccagcctgggcaacatagcaagacctcgtctctacaaaaaaatttttttaaattaaccaagcatggtggtttgtgcctgtagtcctagctacttgggagactgagccaggaggaccccttgagcccaggagttggagcctgcagtgaactatgatggcgtcactgcacttcagcttgggtgacagcaagacagtgtctctaaaaacaaaacaaaacagttcaggacctgttttgccaaaactgacagagccttgcattgctgaccctacattttacttagaaaacataaatcattcatcgcaccatctaccacctcctcaacagcatcatgtactctatgattggtcattagaattctaaggaaagagaaatagtaattttttttcctctattgattattgagaacttcagtaataaaaatgaccaacaggagaataatttagaagaaatagttccttggcttgcttattttctaaactgtacctttaagagaaaggctgaccacccgtgtttccccaagtcaaatcctgaatggaccaaagaggagcccttaagcgtatgttcagattgattgcttatggcataaataatttaaagaaatttaatggattgattcccttttaaagtttcatgggaggaggttttataatccttcatcattgatcattcttgctatgtaccatttgcttgtaagcaaaaaataagagtttggcccggcgtggtggctcatgcctgtaatcccagcactttgggaggccgaggcaggcagataacttgaggtcaggagttcgagaccagcatggccaccgtggtgaaaccccgtctctactaaaaatacaaaaattagctggctgtggttgctcatgcctatgatcccagctacttgggaggagaatggcttgaacccgagagacggaggttgcggtgagctgagatcgtgccactgcactccagcctgggtaacagagcaagattccatctcaaaaaaaaaaaaaaaaaaaaaaaaaaaatatatatatatatatatatatatatattaataagagtttggatacacttatgagcatttgtgcccaatattcgggtcattttgggaacttcccttttgaagagaaccagtttaatggtgttttgtcttgcagagctctgtgcagagctcccaagctggctgctcttgagctatagttagcctatagtttatttgttccacagttatttttcaagtttttttaaattagttgccaacattttaaaaatcaaaattatttcacataaaacgcctgaatgtttttacttttcttgaaagttggaatatcaagaaaagctagcctacatttcacatggcaccctttggctggagctgagttgttgctgtctcctccatacaggtcatgtgccttccagttcagtaagatcccaccaccataccccaacttagtatttgaaggggtaacccctgctcaatagtcacttgttgctttgggtcccaaacagaatgatgttccccaagctcagagctgacagctcttggttactgctaaaaatcagttcaactgtccaagtaccattttctaccactgagtatcccagtaaaagtatttaaaaaaaaaaatgtgctaaaggctcagagaccatctctgaaagaggaaggccaggaggagtatctgagtttccactggaggggcatatcacctctctgctgacagttttaggaggtacaaacttttgtgaatgtatagattctggtgtagtcacacaaaaagagtcttactaagaatagagaaataggctgggtacagtggttgacacctataatcccaactctttgggaggctgagacaggagaatcacttgagcctaggagttgaaggccagcctgagcaacatagcgagacccctatctctacagaaaattaaaaaattagctgggcatgaacctgttatcttagctacttgggaagcagaggcaggaggatcatctgggcccaggaattcaagcttgcagaggctgtgatcataccgctgcactccagcctgggcaacagagcaagaccctgtctgttaataataataataataataataataataataatagtagacaaagtttcaagttgagacgatgagaattgctgtaatagataaggggatcctgacatctagtggacaacattatatactgtcgacagaaaaaacagcccatgacaaggaaggttacattttttctttttacttgagaaaaacacagttataacctctttttgttgttgttgttgttgttgttgtttggagttcattatcttggaatgcttttttggaactcaataggtagattttgatgtttctcaatgaggctttcatatgtaaatgtttggctccatcttattttttatttcctttttagaccaattcaattctgaatgtgcaaaagacactattgactcttacattataccttctcattgaaggatggataatttggtatgaaaatcctctgcagattctggaggaaattgctaatgttttgattacctttagcagaaaaataacaatgtatcgctatcaggagagggatttctggggaggggacttaacatggaaattataaattcattcatgacttttcttttttaaaattaggctttctcttgtttctcagaggatttatcaattatgcaaaagttcggaaaatgccagaaactttctcaaaatctcctcaggaccagagttctctttatttattaaaggtattaaagaaaaaatttattctagtcccagtgtaaccatgtgctacattgtagatttctgttcagttttagagtaagtatctgtgataggggcatatagagaaagtaaagaaggcacagtccctgccttttagcagtttcttgtctagtaacagggatttttacaaaaagctattgataaagtcaaattagaaaaactgccataggagaaatcattacttcatgctttgacagtgtgaaagaggcaggggtcttagcagtcatcagaaagctgggatatttggctgcttgactgaattccagcatcctgaagccactgcttactgttgcaccagttccagaggatgacattcagaaggaacccttcaggcctaatcactcatctctgcccctaaatcccagaacctggagagactgtgtagtactttaagaactccttttgtctgggctgctgacaagcccaccaagtcttccatcttttaaaattgtcattgaggctttccccatggctttcttcaggttgacattctgataccactctaatgtgaggaaggtcaaactcaagaaaagaggagaaaggaccttttcccttgcctagaacaggaggactaagccctgccaaggctctttgattcacacatgattttccccagaggctgacactccatcttacatcaatggctgcctgaaggaagaggtagagaatcttgagaaagagtacaccagagagagctctctgtggtagcaagttctaataggctttcttggacttggctgtttgttccttggggctttggggactgtgttaatcatagagtggaaccttgagcaccacctagaggccagtccagttgaaggtcttaggcaaagcattataaaggataggtggtgtagttcagataaactttacatgatagtggagagtgtggggaagaatttcagaatcatctgaggcagagtgaagagcaaaaatagggaagtatggggcttgttcagaggatagcagcttgattggggcataaaggagtattgagggtcaaggctaaaaagtcaggttgaaacaagtttctcagataccttgagcaccaagcccatttgcagtagaaccatgaaattgatagagccagcgacatgttgggtcaatgcaagactaagcccacgtggagcacgaatttgttctgtactcgcagagtgtcagtttatataggcaatcatatactaatcgtgaggttctcaattttgaagacttgtggtggtctcaggaggtatgcttcaagaaaataaagcatctaccatagttgattttccaggtgatagaacagcattgaaagcttttaggcaacagaatcatataatgagaccctcttgaatataacaaacattttttcaagcatagtatagtaaactaaaattaacactcatggagttttccatgtgtcagataatatattaagcattttacatggataattaaatgtatgtaggtaaagaaggcttaaactgcttttaaagttagtttcattattaaaaaaactagtattcattattaaaaaacaggaatctttgattgcatgtctgaaaatgtgtagcatgtcatgaatttgaattcagtcacacagtctggacacagatggatattcataccactggcctgggccttaatgcctacaatgtgtctttgtttatgtctagaacataagagacttattagatattctttgcagttttatttagttaccattgtcatctttcatctatatatcagattggaatttatagttagccttaggtttaacttttacctatcaaggattccaaagctagtactgttgggttgctacaagtcctccttatgaagggtgctgaaatcacctaatattttccctctaatacattgacacccaagatatactgaggggacagatttctttctctttttaaaaaaagtcatcaattctgtaggtgtagtctaatgttagctctgaatttctaaatggactgagatcacacctctgtctattctctttgttcttcagttagaaagaaatctgaatctttattaggggattgcataccttcttcatcttggttctaagcataagaaatagcagcccagttgacagaaagcctgaacgtctcagttttcctgtgttgtttggtgctttactgaaaattttgttgttattgtttggattttctaaaatttctgaaatgttttttgatattctaaatttaaaactatgtatgtggggttgtgatttgaagtaggcagtccttagtaataataatgttgtaggaagtactaagggagattcaagtgttgagcctgctggtgcttcaggagtgtacgttaagagttgtcagaaactttctcagaggtaaatgaatgcacaccattggagggcataatagagtttatttgcttttctatttaactgtgataccctggtttatttgaaatcattcctgacacacccagagaattcagagctctaaattaagggtttgaggaacttacagcagaatttagaattctaatgacttcactaattttccagggatccccagagggaacctgaaaagccataatatcctatgggactgagttccctttgtccactgaaagggtaagaactctgtccagatccagatgtagaatcccttctttctctagttcagaggacatttagtttaattttgttagaagacatactagaaaaccgtcatgtgaagtaagaaagctttatttttttcctccctatcgattctttattatgcttatcacagtttttaaaaagtaattatctgctaattccaacatcaggttagataagtctatttgtattgatggatttttctcttgactctgggttacattttcctgtttttacacacctggccagttatttctttgttgcagcattgttaaattatcatgttgtagagattatgagctctgaattttgtcagaaggttgttgagtttttgcctagcaggcagtcaaattactaaaggataaatttgatcttatttctaactgtcatgtacagtgattcttctagggagatagggataggggagagggaaaatgtagtttaaaaatcctaggctaactcctgctgaggaaaatcactggtaaactagtgactactctgctaaattgcctctcttgacattgtcaacctgaagccatcaaaaggatcagaatacagagtttatgcaagtagaaagctgggaatggccaatcagagaacacaaactttagaaaatggggtcagtgttcccaaaagctttattttcttttatggacttctacttctttgatggagatgtcaccaattaaaaggtagctgaagaaagacacaggtagggaatttttaaaataactttgccagaaagatacagatacagaatttttaaaataactttaccaaaacagaaaaataatatactttccatattgtactaagatttggcattttactagaaaagggcataagaatagtgacttttctagtcaggctacctgaaatatagtacaaataaaccaatcttgaagtgatcccaaaacacctccttctaatcagtcaaagtagatattacaaaagctaaacattaaagctcatgtactactatcataggggcagctctcaggttccttttcagagtccatttaagcttaagggagggaaaaatgtattaataagctgttatctgttttcctacttcataacaaaatgccagaaggaactgctaaaggtgtgcacagtggccatcaagaagtatgtgactgggccgggcgcagtggctcacgcctgtaatcccagcactttgggaggccgaggtgggcaggtcacctgaggttagaagtttgagaccagcctgggcaacatgacaaaaccccgtctctactaaagatacaaaaattatctgggtgtggtggtgtgcacctgtaatcccagctacttgggaggctgaggcagaagaatcgcttgaacccaggaggtggaggttgcagtgagccaagatcacaccactgcactccagcctgggcgacagagcgagactctgtctcaaaaaaaaaaaaaaaaaaaaaaaaaaagtatgtgactgaggaagcagaagaaacagatacctgttataaacatctgtacctgaaatggtaaaaaatgtaaagtgtttatacttttcttatttaccagttcactggaaaatacatttttatggattaccacctcctctgatgctgccaggtgaatgaggagttagctacagtaataaggtggtaacacagttgattggttatatctaacactatttgtgtggtaccaaaagcacttgtacatatgaagcataatatattttaaaattatttattttaactgcatctgaaatagaaggaaaaaaggaggtttttgagccctagcattaatgagctagggccttcataattaaaaaagtgacctagtgagctgtacctattaggcatcaaatattttaatttttttccatgtgataacagacactttcatttctccttttctgtaatttttctcctgttccttaatacttcttttattttattaaaggaaattcatctcaaggtaaaggtttacttattggactatgttataagatagcaagataatagggaaatggtgtttccagtagaatttttgctaaacaaaatttacatgtctccaagaagaactggttgtcttcctcttggaattaaatgatctgtaggctattggattcctttttttttttttttttttttacaaagtgatctgatctataatttagtaatacggtccagagtccctaagatctgtttgttcttcagtttgtgtactgccagcatttctgtaacactgccactcaagcctccaagtctcagacagcagtgccctctagcagtctttcatttggagattgtgtggtgattgagattacccactggtgacctctctaggtcctcttgagatattttattttaatttaatttttacttctcttactgaacaagcaaagggatttttaatgtttgttgggtgtcaggctttgactttgagggatctattaaagctgtttacctatgctaaagcatcataaactaaggaagtggccaggtaattgtccgtaccatattgtatattcttgcaaactctgttttgacctactagtcactgtttcatcagttttcatctattaaaagaaactgggccaggcatggtggctcacgcctgtaatcccagcactttgggaagccaaggcgggcagatcacgaggtcaggagatcaagaccatcctggctaacacagtgaaaccccgtctctactaaaaatacaaaaaatcagctgggtgtggtggcgggcgcctgtagtcccagctactcgggaggctgaggcaggagaacagcatgaacccggggggcggagcttgcagtgagccgaaattgtgccactgcactccagcctgggtgatagagcgggactccgtctcaaaaaaaaaaaaaaaaaaaatatatagaaactgataaaacaaattatttatgtttataatgagtctggcctcattgcaaaaacattcatttaatattcatctgccaattttcttttttgcttctgcttggcttttagagttcctcatatttttgtgtctctgtctctgtgctttccgaggtctttcaatctcttatgtctctcaatctaaagaaaagatgtccttgtatcagtttcagtggtttacttcaaatcacaacacaaatgaatttatagtttggaaaaatgttgataatattaaaatatgttcatttcctccagtgtgtttgtagtctctctccattttggcagccttttaaatacttacgcagtaatctaatcagaaaagatacagaagacaccgggcatagtggtgcacgcctgttagtgccagtcacttgcggggctgaggcagaaggttggcttgagccctggaggtgtaggttgcagtgagccgagctcgcgccactgcactccagcctgagtgacagactcttgtctccaaaaaaaaaaaaaagaaaagaaaagaaagaaaagacatagaagagtttcttgtggcttttatagtaatttggaggatagaaaagtaagttataggccgatatttgagcccatccaactctcacttcttgctgagtaccacaaaatagtttctcaataaatgtttattaagcagtaaatgtatctctttagtgtttcttcttttattcttaatagttcttcttttattgttaatatagggaattacttgtagtttttcccaagttattttcatgagaaccaaaacaatatactagtacagaaatgcctcaaagtattagatgaaaactgcatttagtaaaatgcgtaggcattatttcgagagcatttatttcagtcagatatattcctttcttaaaatttagtttgaaagtacatgtattattttaagagaaacatgccccaaatacaatgactttttaggaaaacttgttttttaaaagtatttgtggtacctgtgagtttatccttgttttctaattttgccatgctcggttgaggtatagcatgacatggttggcatccttactggaaagcttccttgtgcaatatctagtggaattgttagggtaaacagtatcttcaggtatattaggaaaattacttgtgcacttggattatgctctctgtcccaagtttcagtcattgctgacagcgttcagtgtgggatgcgcagtagcactgcttccattgtattcttttgttaagaggtttggaaagagagactcctgacttcaggagtttttagaaaaagcattccagttttttctagttctagaggccttgataactttaagaactgaaattttaatgtttattgaataccgtgatgtcagtatacagtaatcttaaaactatgttaaaatgcacttgtgtctccaaacatcctgccttcttttgtgactacatccttctcttagccaaaataacaagctaactagagtttcaatattcaatatccttctctggcagatgttttctggcaaaggccttcctgcatttatgaattctctctcaagaagcaagagaacacctgcaggaagtgaatcaagatgcagaacacagaggaataatcacctgctttaaaaaaataaagtactgttgaaaagatcatttctctctatttgttcctaggtgtaaaattttaatagttaatgcagaattctgtaatcattgaatcattagtggttaatgtttgaaaaagctcttgcaatcaagtctgtgatgtattaataatgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgctatgactgtgtttttgcacataatccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctatgttaaaactgtaaatacaacagaacattaataaatatctcttgtgtagcaccttttactgtagattagtgcttaatttcttggcttgcatttgttgattgctaaggcaattttttctaatcttagggaatcattcagtagatgcgattaaaaaactaatgttgggtcaatttttttcttcattttcagcacaagaagtcctcttatatcctactaaatacattcctaaaaatgtatttgaacattggttctgtaaaagataatggactaaaaaagtagagaggagttgtagagatcttaaatcattctggaattcctaattatgcttcaatttttagacataattttagataatttatttccagtgttttctgcatgttctcatttgttctttttctcagttgaatgcaccaactggtttgagtcctgtgagcattcagtcagttgaaattaaagattcctcatttctcctgatttctattcttgtctcaatcttaaatttagagaccagttgtttttatgatatcagccatttgatttttttcattttctatttaagaaatatgaagaaaaaatacaccaagatggtcaaattactacacaaatcagcaccagcacagtctgatagctgcaaatgtccattcatctgctgtgtatgtatatccagaatcagcataggaagtcgttcaggatatcagtatataatgcacagaagtgtgggttgtttgaaagccaaacaggaaaattaggagcctcctggattgacatttcaatgatccctctaaccagtttatggattattatgaataatagtgtagtgtgttctttttcagaagttatatttgataatagagaagggagttttatggaagtttctttgaagatttttttttttccatttcgaatcagattatagcaacaatggagtttggaagtttgtatggcctataatgttctaagttccagaatgaaaagatctgtaacaatctgaatagatgtggacacatatagcagagagaactatgtaaattatcttgcagaacaaaatagaagggtcctaaatcacgttaactcaaacattgtagactagctttgtgtttattcttcaggtccttgcgccttatttggttttgtatattcaacgaactgaaatatttggaattcctatttctacgtatttggtggtccataagactttgtcaaatgtaaacctacagtttgatacgctttaaaatacctagttaagaggatgatttctctttaatcgtttaaatgttctgaaaattaaaatcttttgaggcacatgaagtgggcaccatatatcatctagagtccttactggtattcaggatgaaaatgttcacgctgcattaattgtcatttttctctcccatgttctttctcactttgatacgttaatactgataatggataaagagtgagtttttataataaatggttttggaaaggtattcataggaaccgcggttatttacttaaggttatggagtaaactagcttggaccttgggctgcaggacgactaggattcacccataacgacacagtgccctatgtttcttaacttcttgttgccatttgaaactctgtactcttatgtttaaagggttctgtatagccattttttttttcagaaagttacattgctttgtatagaaataaaaggcattattaaaatttgcttgttaaaaaatgataagaatgaatatctttacttatagggtcaattttgtttagagttttaaagaaacaacagaagttttagtgaagtaaatgattttttttttccctttgctttcttattacatactgacttgagctctggcccaccgtgagtcattattagtacagatgaaattttgtttggtcttcactatgtgttttttgtttgtttgttttttgagacggagtctcgccctgtcgcccaggctggaatgcagtgttgcgatctccactcactgcaacctccgcctcccaggttcaagcgattctcctgccccagcctcccaagtagctgggattacaggcgcgcgccactacgcccagctaatttttatatttttagtagagacggggtttcactatgttggtcaggctggtctcgagctcttgacctcgtgatccgcccgcctcggcctcccaaagtgctgggattacaggtgtgagccaccgcacccagccttcactgtgttcttatactaaaatatactctacttagctataaatggtttacattcaaaggaagaagtaagataactactgaagtagagtatcaggaccaatagaagtgatatttcaatttgagtgattgcctgagggggtaagattctggggtgagaatctagaccctaacaaaactagacccctggcctcaggctttacatccagtccaatgccctcttcccattgtcccaaccttctggagggttggcgtgtaaacattgacataaaatgaaaaaaatcatttgaccgtgcttcttgtaaacatcagtaatctaattttaactgaaaacatgctaagaatagacatggaattgtacatttatgtaaagtaggaggatgtgtgtttgtatagatagatggatgcatagattttttttttttttttttttttttttttgagacagggtctcgctctgttgcccagacttaagtacaaagtcgtgatcttggctcactgcaaccttcaccttccaggctcaagccatcccacctcagcctccccaagtagctgggactacaggcatgtgccaccacacccagttaatttttgtatttttagtagagacagggttttgccatgttgcccagactggtcttgaattcctgggctcaagcaatctgaagtgctggaattacaggcgtgagccacctcacctggccagtaggagggtatattttatgaaaaagtagttgtgacccctattacctatgtgaatctggttcttggttaaacctcagtggtgaatataggttaatcatgtgcaaaatgtataataattttcaggttctgtgggtggttcagctgttcagaagattttcacctctcaggcttttagaatatgagttgaggaatgatgagggtcataacagaaggcaaaatatgcttccatagatggtttatatatttaaaaaaaatgtttttttgcgtattttaaaccctaaatagtgtttctatcctgtaagatggataaggtggattgggtgagagcatttgttaaagcagtagtacagtactcttgcatctccttttcccttttgcgtatacctgtacacgtttgtgtatgtgtataattgtgttcttttagactacctacagaaagtgccctttgacaagtagcctctgcatgttggttctttgtaggctctactgttaatgtggaagacatctcaaagaaatacactgggaattcccaaaggggagagtaagtttaggttgaaagtgaaagaggtgttcaggaggaaatgatggttgtagaaattgatttttgtatcatctccattctaaaaagaagctattttataacccagttgattgtatcacctacaactttatggcctatagaaagaactgctcttacgtaaagtacatgaattgagagctgtcctcccgaagccagtttccctgactataaaatctgaaatagtccatctactgcgtggcattttaatcccctcagtcctccattattctcagaattttccgcttataggataaatacattactcaaaggttttgtgttaatttttatacctgtttttacttatagcctgtttctttttttctttaccaaaaagcatcgtcttctgattttgagagttttaattcttaagtatcagttgatgaattaataaaatgtaaatgtaaatgcacatctcttaaacctgaatacagaacatattaatatagaggattctcctacatttatgcaggattttacctaggtcagaaactgagatgcaattgtgtgaaatagatgtcgtccaacctgctttgtatgttgttcattctgctctaaaaacagctaattttaatcattgatgtaggaaataaatgtttcacagacttttccccattcattagtgattgcagattcatagacatcaaaactgagaaaagtggtttcctctaagccttaacaaagattagtgctggagctctgggaagatggcagtattcattgttattcttcagttgtctggaaggttcgcgagtgatatacgcagcctgggaaacaagaaaacaggtcaaaaatctaaaagaaatctctaaaatagagaagatgatttccctgacaaagcattattagaaaaagtataaaactaaggaagttctgccttctggttatgaaacccagcctgattctaatgaaattcaaccaaaatgccaaagctagagtgattttcaccttgcatagttgggtttgcattcgtgtcccctgctgagagctgctttgcctcccacttcccctcccccatttaagccagatacttcctgagccaagttggagaggactggtgcaacctatcatcataactgggctagagttactcttgatgaaacatccctcttttcatttgcagaactgtcagaaatgcctaaaatgctgcttaagtgaggttttagaaagaattttgccatctgcttcctgattccacatctttattttgacagaacatttcataggccgtatttggcagttcctctgtttggatgtcattttaatatgttgcttttttctctgattatcaacactgaggattttcatgtatatgatttatttccagtttatattttaacagattgcagataaatcagtgattgcagagaaatcagatggatcaacattttcactatttattactttttatgtatttattactatttatttattactagtatgctttggtactatatagttgatgaaaatagtgggcactggtgacctgaagaagcaaaattagcagtccatatttttataggtttgattcagtgactctcaagccctag

SEQ ID NO: 7 - DNA, 6564 bp, Homo sapiens
atctgtatatgcatggtcatgtgtaggacaaaaatcatctttttttctctttttggttaattatatattttgtccctgagactgtgaagtgagttgaatctgtataacctatttttgtccgtaagctcttctctggtttagcagttctctagatttaggtattggacttatatatatatttttccaatatccacctcattgaattatagacttagttcctacagtagttccacagagcttccaaataagggtaaggataatgggtaagtaagtacttacctctcatcttcacttccatagaagaaggctgagtgcagtacatttgtatctacactttataaattatgaactagtttttcatcaaaaaatttcatggatgccaggcacggtggatctcgcctgtaatcccaatactttgggaggccaaggtgggcagatcacctgaggtcaggagttcgagaccagcctggccaacatggtgaaacctctgctctactaaaaatacaaaaattagccgggtgtggtggtgcgtgcctgtaatccccagctacttgggaggccgaggcaggagaatcacttgaactcagaggcggaggttgcagtgagccgagattgtgccactccagcctggacaacagagtgaaactccatctttaaaaaaaaaaaaaaaaaaaaattgatggttgggtagattaagaattattttgtctttatataggtctcaaagctcatggccacaatattttcctcttcagaatggcaggactatgggaaggaatgtgcattcagaacaacggggaaaggctatagggaaattaatttcaaaatatttggctgagtgattgtggactcttccttggaagtctttaaacacagaatagatcctcctatttggaatgacttaagggtaaaggaaaaggaatggattagcaggcgccttaaagaccccaagaactttagagatttgtatgtggcccatgtcagtgacagatggaacagtaagttcaatagacgtcatgtctgccgctcccaggctatgcttcctttaatgtagtgaccttctgggagcattgtcctcaattgtctgcaagagggaaaggagtacatctaaatttctggtaaacttaatttgggtggcttattacctactgttatgtatcttttaaagatccagtgatccaaaaagctgtgctttgcccaaaattccaaaatgagaaaaccataaaaaccaccctctagtttctggagccattttcaaatctgtgttgattctgaaggaatttcatttacgtagcattttcacatgttaacagtccttcccctaagggattatgggtagaggaaagaagatgaggtggtaaggaggcactggaggagatgaagtctgatttgtttagaaaggaccgatggaggagtgtagcagacactccttttgttctttgctcctgctttaatccttcagacccacaggacaagattgccagctaattgcaaggagcccgtaaaagagaattggcctaaattgaagaataaatatattttttttttaattcttcctttatttgggtcactttgctttacatatgtttatgtctccaaaaagtggttggataccctcttgagaggttttctttacaaaaaagaaagaaaaaaaaatctgaaattccaaagcctctttccctgccattgtagcacttaggcaaaagcatgaagaactaaatccactagactgtagtttaaacttgaaaaaacaaaaacaaaaaaccccaaaaagactataatgtgctttagagtggcatttttatacaagtgactcattgctagcagtatattgaccattttaaagtgcaatctgatctttcatgggtagacatttaaattctctgagtacagacactgtacttgggacaaattattatatttgtaccttggtgccaatcttaatcatgtactcattgggtgcttattttcaaatacctgatccttctaaaaacagtatactaaaacctgttgttaaacatatatcaagtttaatagcaagccatggatactttttactgccttttatttattgctgggtgtattttattagaaggaaaggtaagtaaatgtgttttgcagaaaatatgtagtataagtcagctccagtagcagttattattttttaactagatggcggagcatttacacacttagtttattagtgaccttcccataaaatttttaggaacacagggccaaacagaggtcataagtttatccaagccaacccacttctttggtgacatcagacaactgaagagaagccaagaaaacgttccctccatcttcaaattctatgtgtccttggctttgcctccatccatagtggtttattcccctcagaaagaattgcagatggaatttgtataatcacctgtgttagaagtgatgatctactttgaaaaatacagtttttatgtattataaataattgtgctctgccttcttgcatatagttgctttgggcctttatttgataaaaactatcttattagccagttatgacatacatgactttatgctggatgtagtatatacaaagatttgtacaagacagcccctgaacacaaggaggttttagtctgggaggagattggtaggagacaaagtgctttcctgtccaagaaatcttaatatgtaatgtttttctgcttcgtttgcatagtgatggaagaagctttttacacttttcaagcttgctgctaacttaattttcaccagaagggcacttttaaagatgttcgtaaaaccaaatgccctcaaaggagagattactgccacttgaagaggccacccccaaaagggccaaaaggatgggggaacatctcagggtgctatgtggccaggacctagaaaaccagctgccaaggggacttgtgactgttagcctagggcttgagccctcgctccccatttactggctgtgtaatcttgggtcatttgcatgtcctatccaagctttggttttctaatctgtgtgctggaatattttctcacagggccatttttagggaggtaaagtgcccagcatctagcaaacagctcttcacagctgatcagctatagtattgggtctaagggcaggacatgaaaagcccaaacctataattcaaaccgagggaattcatttccttttcaggaccgtatagctctcataaaccctgcctttcctcattccttctttcctaaatgtcattaggccatccctgataaatcagatcattcttatcctcctaaattcctgcagcccagatgagaatatgatttactattgcttttctggtaatattatcatatgtttatgtgggaacttatatacatgttcatttgatcttcacagattactataaaagggattattttgcgttctttaccaccaagcaaccaaagcctccctcccccggcaaataattttccaacactcactaacatcaaaacactctacagattcaataaactttgattctcatttcctttcctgagttctataggctgaaactctggcctcttttttgcttagtccatttgtattacattacaagacttgccgtatactatctttgtatggatttaggtattcgaagcagcctttgaacttcttatcctttgctttgtttctttctgcactggccttacattctctgctcttttccctgccctttccatgtcttctttgccgccctctcgacacctcccccttcccctacgtttccagtgctgcttagatcactcttgttacttctctgtcctaaattcctacacaactgtagatagggccattttctcagtggccactctccctttcctttctccaaatcttaaccctgggccatttgaaccctagttacttcttttttttgttttgttttggttttttggtttgtttgttttgagacgaagtctcactcttgtccccaggctggagtgcaatggcgtgatcttgactcactgcaacctccgactcctgggttcacgtggttctcctgcctcagcctcccgagtagctgggattacaggcgcctgccaccgtgcccggctaacttttgtatttttagtagagatgggggtttcaccatgttggtgaggctggtctcgaactcctgacctcaggtgatctgcccgccttggcctcccaaagtgctaggattacaggcgtgagctaccgcgcccggcctaaccctagttgctttctatcttcccattccatctagccgcatccaacaagaaaaccacatgcctgctttctcttcctgaagctctccaaggaacgttcctgtatgcccctggatagatggttccagcctgtgtctgtgccccccaacccagtcctgccaatccaagcctgcccagatagggcctgcctgttttcccctttattgggggatctggggagcttttcacagtggtggcgtttgtcacagtgttcacaggctcacctcttcatgccccttcagtactagtttggcttggccattaaacagaaagcattggggtgtcctctgctctttgttctctctcactatatagatcacctccaatggcatctgcatgaatctatgacaactctcgggcctccatggagaagcacctggctagtcccaaatttcataatccttctgatgcttctgaattaccttccacaaaacactaggattctataaacttttaattggaattttatcttttaaaaacagccttaatcatgtaataagatgtattttcaaatatgtgttcaattgtgtggttatttgtgatctcatttacagacatgaacaaatactaaaacaatatataaataaatttactcagagtatttgggatctcagattttgttttacaaaaagggttctgcagctgaaaaaaaaatggcttgaaaaccactgccctaagtaaacactcatctcttactttgcctaaaactgttagaccgttatacattttttttcctccgtcaatcttacaggggtttcaaaatgccctgattttgcaaaacttatgagcacatctaaaaagtcactttctaagcctactatctccaaaaatcaactgtttgttctttttcagacttgtttttgtgaatttattatttttacataggtagtactagcatactggcacagcccagtgagatgaggtctcttaagccattctgtttatatcagatgttaaattttttaaatagtctttgctgcagtgtagattatcttgtctttttctgcttttcagctcgttctgacaaatgccccatgatgtttttgtttttgttttttgagactgagtttcactcttgttgcccaggctggaatgcaatggtgccttctcggctcactgcaacctccacttccctggttcaagtgattcttctgcctcagtctcccgtgtagctgggattataggcgcccgtcaccacactcggctaatttttatatgtttagtagagacggagcttcaccattttggccaggttggtctcgaactcctgacctcaagcgatccgccctcctcggcctccccaagtgctgggattacaggtgtgagccaccacaactggcctcgatgattttttttttttttttttttgagaaggagtttcgctctgtcgtccaggctggagtgcagtggcgcaatctcggctcactgcaagctccgcctccggagttaacgccattctcctgcctcagcctcctgagtagctgggactacaggcgcccgctaccacactcggctaattttttgtatttttagtagagacggggtttcaccgtggtctcaatctcctgacctcgtgatccgcccgcctcggcgtcccaaagtgctaggattacaggcttgagccaccgcgcccagccgatgatggtgtttttaaagccaacttctaatacctcttttttccattgcttcattgccaagaggaataattagactgttgcaacaattatgacaaaggcgtcctatacccttggtcttctttcatccatagggcatccaaaagagacctggtttctctgttgattggaagttcacatcaaattccttgaccctgggattaaacctacagtaaacatgtgtatttttgtgttgcagatgcgtatcatagatggatagtaatgaataccatttattgagcatttatcatgtgcgaagtactctgctttcttcttcacaggtagatttaatcatctccccaaccctttgaggtagggaatactgtgatccttgttgtatatttttaaaaaaatggagactcatgttaactgacttgccccttaccgtccttaagtggggaggctgggattaatttggtttgtgttgttagagcatgaactctttaacaacaatagaacactcatcatctttcctgtgtgtgcacctgcttccatttctttttattctcctgtcccttcttgatctacactttaacacattcctttctcttcccacaggattccctttattgaaggtgggaatgagggggattctggactgcaggcacaggacatctgccctcccacagcagcctga

SEQ ID NO: 8 - DNA, 24 bp, Homo sapiens
agaacacagaggaataatcacctg

SEQ ID NO: 9 - DNA, 22 bp, Homo sapiens
tgcactatgattttgcttccag

SEQ ID NO: 10 - DNA, 25 bp, Homo sapiens
cattttaagtagcatgagccatgtc

SEQ ID NO: 11 - DNA, 23 bp, Homo sapiens
cttgccttaggcttatctccctt

SEQ ID NO: 12 - DNA, 22 bp, Homo sapiens
gcctcggaatgtcagctacttt

SEQ ID NO: 13 - DNA, 19 bp, Homo sapiens
ggtcatctggtgcctttgg

SEQ ID NO: 14 - DNA, 22 bp, Homo sapiens
ccagcctaacaatgctctcctt

SEQ ID NO: 15 - DNA, 21 bp, Homo sapiens
cggtgcttacagcctgagaag

SEQ ID NO: 16 - DNA, 16 bp, Homo sapiens
aacgccaacgccatgg

SEQ ID NO: 17 - DNA, 25 bp, Homo sapiens
ttaatgtttgaaaaagctcttgcaa

SEQ ID NO: 18 - DNA, 26 bp, Homo sapiens
tgacaacacctttagaatcccttagg

SEQ ID NO: 19 - DNA, 25 bp, Homo sapiens
cttcaccccttagtagctctgtgtc

SEQ ID NO: 20 - DNA, 24 bp, Homo sapiens
aatccttgagaactgcttggatct

SEQ ID NO: 21 - DNA, 20 bp, Homo sapiens
ccctttgcacctgttcctgt

SEQ ID NO: 22 - DNA, 17 bp, Homo sapiens
tccggtctgccaccatg

SEQ ID NO: 23 - DNA, 31 bp, Homo sapiens
aaatgtcagatagcaagtattatagggtttg

SEQ ID NO: 24 - DNA, 20 bp, Homo sapiens
tgccaagcactgagcttcac

SEQ ID NO: 25 - DNA, 19 bp, Homo sapiens
aaccacgattctgctgcca

SEQ ID NO: 26 - DNA, 15 bp, Homo sapiens
gccccacccccgact

SEQ ID NO: 27 - DNA, 19 bp, Homo sapiens
acacttgagcctcggaggc

SEQ ID NO: 28 - DNA, 19 bp, Homo sapiens
cagggaatcagagcgagcg

SEQ ID NO: 29 - DNA, 16 bp, Homo sapiens
cccgcctcttccccag

SEQ ID NO: 30 - DNA, 14 bp, Homo sapiens
agccccggcgtcga

SEQ ID NO: 31 - DNA, 17 bp, Homo sapiens
tgtcgactgcgccatgg

SEQ ID NO: 32 - DNA, 15 bp, Homo sapiens
ccgcctgtgggtcgc

SEQ ID NO: 33 - DNA, 18 bp, Homo sapiens
gcctcaggcctctctggc

SEQ ID NO: 34 - DNA, 19 bp, Homo sapiens
gagctatcggcagcagcag

SEQ ID NO: 35 - DNA, 19 bp, Homo sapiens
tccctgagtcagaacccgc

SEQ ID NO: 36 - DNA, 18 bp, Homo sapiens
aatacacccggcacacgc

SEQ ID NO: 37 - DNA, 17 bp, Homo sapiens
cccctcgcctccgtttt

SEQ ID NO: 38 - DNA, 31 bp, Homo sapiens
ccaccattactgtatgttagattaacacttc

SEQ ID NO: 39 - DNA, 27 bp, Homo sapiens
tcattttctcgggtttgatttaataca

SEQ ID NO: 40 - DNA, 17 bp, Homo sapiens
aaaccagtaaccggccg

SEQ ID NO: 41 - DNA, 29 bp, Homo sapiens
tccatagaataacttcctgggagtagaat

SEQ ID NO: 42 - DNA, 25 bp, Homo sapiens
ttgtaacctttcttccaactgcttc

SEQ ID NO: 43 - DNA, 31 bp, Homo sapiens
tggttcaattgttatgtatgtactcttaagg

SEQ ID NO: 44 - DNA, 33 bp, Homo sapiens
aaaattagctctactttcactaatcaatacaat

SEQ ID NO: 45 - DNA, 29 bp, Homo sapiens
gaattttaaacacacattgagcctttaat

SEQ ID NO: 46 - DNA, 26 bp, Homo sapiens
ccagtaaaaggcaccaaataaacata

SEQ ID NO: 47 - DNA, 21 bp, Homo sapiens
ggcaggtgtaaggcactcttg

SEQ ID NO: 48 - DNA, 20 bp, Homo sapiens
tgtgcatgaaccatttgcaa

SEQ ID NO: 49 - DNA, 25 bp, Homo sapiens
gcattttgtgattttgtgtaccctt

SEQ ID NO: 50 - DNA, 26 bp, Homo sapiens
ttaggaagaacctacaaccacaaaga

SEQ ID NO: 51 - DNA, 26 bp, Homo sapiens
cagattctggaggaaattgctaatgt

SEQ ID NO: 52 - DNA, 26 bp, Homo sapiens
gggactgtgccttctttactttctct

SEQ ID NO: 53 - DNA, 31 bp, Homo sapiens
ttctcttagccaaaataacaagctaactaga

SEQ ID NO: 54 - DNA, 26 bp, Homo sapiens
ggacatggctcatgctacttaaaatg

SEQ ID NO: 55 - DNA, 30 bp, Homo sapiens
gatcatttctctctatttgttcctaggtgt

SEQ ID NO: 56 - DNA, 36 bp, Homo sapiens
catacagaattgaataaatttctagattaacttgaa

SEQ ID NO: 57 - DNA, 22 bp, Homo sapiens
catgtccctgtagtcggtaggg

SEQ ID NO: 58 - DNA, 31 bp, Homo sapiens
gttttatacagtgacaacacctttagaatcc

SEQ ID NO: 59 - DNA, 25 bp, Homo sapiens
aacacctggaagcaaaatcatagtg

SEQ ID NO: 60 - DNA, 27 bp, Homo sapiens
ggatcatctcaaaagttccaagctact

SEQ ID NO: 61 - DNA, 32 bp, Homo sapiens
aattcttgacactctttctataattagcgttc

SEQ ID NO: 62 - DNA, 24 bp, Homo sapiens
atcaacaaatgcaagccaagaaat

SEQ ID NO: 63 - DNA, 24 bp, Homo sapiens
aatgtgttagcagaaaccagtggg

SEQ ID NO: 64 - DNA, 32 bp, Homo sapiens
ctctacaactcctctctacttttttagtccat

SEQ ID NO: 65 - DNA, 29 bp, Homo sapiens
ttgtatggcctataatgttctaagttcca

SEQ ID NO: 66 - DNA, 24 bp, Homo sapiens
ccacttcatgtgcctcaaaagatt

SEQ ID NO: 67 - DNA, 27 bp, Homo sapiens
cgccttatttggttttgtatattcaac

SEQ ID NO: 68 - DNA, 23 bp, Homo sapiens
cgcggttcctatgaatacctttc

SEQ ID NO: 69 - DNA, 27 bp, Homo sapiens
gagtccttactggtattcaggatgaaa

SEQ ID NO: 70 - DNA, 30 bp, Homo sapiens
caagcaaattttaataatgccttttatttc

SEQ ID NO: 71 - DNA, 28 bp, Homo sapiens
gttatggagtaaactagcttggaccttg

SEQ ID NO: 72 - DNA, 29 bp, Homo sapiens
ttcatctgtactaataatgactcacggtg

SEQ ID NO: 73 - DNA, 19 bp, Homo sapiens
gctggaatgcagtgttgcg

SEQ ID NO: 74 - DNA, 27 bp, Homo sapiens
ttgaaatatcacttctattggtcctga

SEQ ID NO: 75 - DNA, 17 bp, Homo sapiens
gacctcgtgatccgccc

SEQ ID NO: 76 - DNA, 23 bp, Homo sapiens
acaagaagcacggtcaaatgatt

SEQ ID NO: 77 - DNA, 22 bp, Homo sapiens
aatttgagtgattgcctgaggg

SEQ ID NO: 78 - DNA, 27 bp, Homo sapiens
gccaagatcacgactttgtacttaagt

SEQ ID NO: 79 - DNA, 23 bp, Homo sapiens
acaaagtcgtgatcttggctcaa

SEQ ID NO: 80 - DNA, 21 bp, Homo sapiens
gctgaaccacccacagaacct

SEQ ID NO: 81 - DNA, 27 bp, Homo sapiens
gttgtgacccctattacctatgtgaat

SEQ ID NO: 82 - DNA, 25 bp, Homo sapiens
cacaaacgtgtacaggtatacgcaa

SEQ ID NO: 83 - DNA, 21 bp, Homo sapiens
ggtggattgggtgagagcatt

SEQ ID NO: 84 - DNA, 29 bp, Homo sapiens
ctataggccataaagttgtaggtgataca

(19) United States
(12) Patent Application Publication
(10) Pub. No.: US 2003/0104521 A1
(43) Pub. Date: Jun. 5, 2003

(54) Disease associated gene

(76) Inventor: Paul A. Whittaker, West Sussex (GB)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 09/902,214

(22) Filed: Jul. 10, 2001

Related U.S. Application Data
(60) Provisional application No. 60/327,554, filed on Jul. 13, 2000.

(51) Int. Cl. C12N 9/00; C07H 21/04; C07K 14/435; C07H 21/02; C12P 21/02; C12N 5/06

(52) U.S. Cl. 435/69.1; 435/183; 435/325; 435/320.1; 536/23.1; 530/350

(57) ABSTRACT

The invention provides a disease-associated gene, the protein molecules encoded by the gene and polymorphic variants thereof, and the use of the gene and polymorphic variants thereof in diagnosis, prognosis and treatment of inflammatory or obstructive airways diseases.

### FILED OF THE INVENTION

The present invention relates to a novel asthma-associated gene, designated AAGB, and to the protein molecule encoded by AAGB. The invention also relates to the use of AAGB polynucleotide sequences for diagnostic and prognostic screening of patient populations and the use of the protein encoded by AAGB as a therapeutic target.

### BACKGROUND OF THE INVENTION

Asthma is a very common lung disease with the following characteristics: airways obstruction—this is usually reversible but often progressive chronic bronchial inflammation—a condition characterised by inflammatory cell infiltration and activation, release of biochemical mediators and structural changes (airway remodelling) bronchial hyperresponsiveness (BHR)—an exaggerated bronchoconstrictor response to a variety of immunologic, biochemical and physical stimuli.

Asthma is characterised clinically by chronic, intermittent airway obstruction with wheezing, coughing and breathlessness. Although asthma is typically associated with an obstructive impairment that is reversible, neither this finding nor any other single test or measure is adequate to diagnose asthma [Guidelines for the diagnosis and development of asthma, 1997, NIH Publication No. 97-4051]. Many diseases are associated with this pattern of abnormality. The patient's pattern of symptoms (along with other information from the patient's medical history) and exclusion of other possible diagnoses also are needed to establish a diagnosis of asthma. Clinical judgement is needed in conducting the assessment for asthma. Patients with asthma are heterogeneous and present signs and symptoms that vary widely from patient to patient as well as within each patient over time.

Many hypotheses have been advanced to explain the pathophysiology of asthma, including problems with airway smooth muscle, the role of inflammation, nervous innervation of the airways and mechanisms related to mediators. Although all of these factors may be important, it is unclear which are the primary (i.e. causative) defects and which are the secondary defects. It is generally agreed, however, that both the environment and genetics are important. Given the multifactorial nature of asthma, one approach to identifying the fundamental mechanisms is to discover asthma susceptibility genes that predispose individuals to develop asthma.

One method which can be used to identify asthma susceptibility genes is positional cloning. In this method, susceptibility genes are localised to a specific region of a human chromosome by using DNA markers to track the inheritance of the genes through families. DNA markers are fragments of DNA with a defined physical location on a chromosome, whose inheritance can be monitored. The closer a DNA marker is to a susceptibility gene, the greater the probability that the marker and the susceptibility gene will be passed together from parent to child. This phenomenon is called genetic linkage. Once linkage to a specific chromosomal region has been obtained, the size of the region is narrowed down using a combination of physical and genetic mapping until the region is small enough to be sequenced and the susceptibility gene can be identified. After identification of the susceptibility gene, any polymorphisms in this gene can be determined and an analysis performed to see whether these mutations occur with greater prevalence in asthmatics compared to non-asthmatics. The major advantages of positional cloning are that it is possible to identify novel genes even though the underlying factors causing the disease are unknown, and the genes identified are of direct pathological relevance (i.e. primary causative defects) because they make carriers directly susceptible to developing the disease.

In recent years a number of academic research groups have provided evidence for the presence of genes important in the regulation of asthmatic and allergic responses on human chromosome 5. In particular, evidence for the presence of susceptibility genes for BHR and elevated serum IgE levels on chromosome 5 in subregion 5q31-5q33 [Meyers et al., Genomics 23:464-470; Postma et al., N. Eng. J. Med. 333:894-900; and Bleecker et al., Clin. Exp. Allergy 25:84-88] was obtained from genetic linkage analysis of 92 Dutch asthma families. Strong evidence for genetic linkage between marker D5S436, raised total serum IgE levels [Meyers et al., Genomics 23: 464-470; Postma et al., N. Eng. J. Med. 333:894-900; and Bleecker et al., Clin. Exp. Allergy 25:84-88] and BHR [Postma et al., N. Eng. J. Med. 333:894-900; and Bleecker et al., Clin. Exp. Allergy 25:84-88] was found in the Dutch families.

No asthma susceptibility gene has yet been identified, so there is a need in the art for the identification of such genes. Identification of asthma susceptibility genes would provide a fundamental understanding of the disease process from which a number of clinically important applications would arise. Susceptibility genes identified may lead to the development of therapeutics (small molecule drugs, antisense molecules, antibody molecules) directly targeted to the gene or protein product of the gene, or may target the biochemical pathway of which the protein product is a part at an upstream or downstream location if the development of such drugs is easier than directly targeting the gene or its protein product. Polynucleotide sequences comprising the gene, sequence variants thereof and protein products thereof may be used to develop a clinical diagnostic test for asthma and for the identification of individuals at high risk for the development of asthma. The results of such tests may also have prognostic value and may be used to predict patients who respond to and those who do not respond to drug therapy. Finally, information about the DNA sequences of asthma susceptibility genes and the amino acid sequences encoded by these genes facilitates large scale production of proteins by recombinant techniques and identification of the tissues/cells naturally producing the proteins. Such sequence information also permits the preparation of antibody substances or other novel binding molecules specifically reactive with the proteins encoded by the susceptibility genes that may be used in modulating the natural ligand/antiligand binding reactions in which the proteins may be involved and for diagnostic purposes.

### SUMMARY OF THE INVENTION

Accordingly, the present invention provides, in one aspect, an isolated polynucleotide, hereinafter alternatively referred to as AAGB, comprising a nucleotide sequence encoding a polypeptide comprising the amino acid sequence of SEQ ID NO:4 or a functionally equivalent variant of said amino acid sequence, i.e. a variant thereof which retains the biological or other functional activity thereof, e.g. a variant which is capable of raising an antibody which binds to a polypeptide comprising the amino acid sequence of SEQ ID NO:4.

### BRIEF DESCRIPTION OF THE DRAWINGS

FIG. 1 shows a graphic display of a region of chromosome 5.

FIG. 2 shows the results of Protean analysis of a putative protein.

FIG. 3 shows the genomic structure of the AAGB gene.

FIG. 4 shows the alignment of SEQ ID NO:4 with other protein sequences.

### DETAILED DESCRIPTION OF THE INVENTION

Terms used herein have the following meanings:

“Isolated” refers to material removed from its original environment.

“Hybridization” or “hybridizes” refers to any process by which a strand of a polynucleotide binds with a complementary strand through base pairing.

“Stringent conditions” refer to experimental conditions which allow up to 20% base pair mismatches, typically two 15 minute washes in 0.1×SSC (15 mM NaCl, 1.5 mM sodium citrate, pH 7.0) at 65° C.

“Homology” or “homologous” refers to a degree of similarity between nucleotide or amino acid sequences, which may be partial or, when sequences are identical, complete.

“Expression vector” refers to a linear or circular DNA molecule which comprises a segment encoding a polypeptide of interest operably linked to additional segments which provide for its transcription.

“Antisense” refers to selective inhibition of protein synthesis through hybridisation of an oligo- or polynucleotide to its complementary sequence in messenger RNA (mRNA) of the target protein. The antisense concept was first proposed by Zamecnik and Stephenson (Proc. Natl. Acad. Sci. USA 75:280-284; Proc. Natl. Acad. Sci. USA 75:285-288) and has subsequently found broad application both as an experimental tool and as a means of generating putative therapeutic molecules (Alama, A., Pharmacol. Res. 36:171-178; Dean, N. M., Biochem. Soc. Trans. 24:623-629; Bennet, C. F., J. Pharmacol. Exp. Ther. 280:988-1000; Crooke, S. T., Antisense Research and Applications, Springer).

The term “variant” as used herein means, in relation to amino acid sequences, an amino acid sequence that is altered by one or more amino acids. The changes may involve amino acid substitution, deletion or insertion. In relation to nucleotide sequences, the term “variant” as used herein means a nucleotide sequence that is altered by one or more nucleotides; the changes may involve nucleotide substitution, deletion or insertion. A preferred functionally equivalent variant of the amino acid sequence SEQ ID NO:4 is one having at least 80%, more preferably at least 90%, and especially more than 95% amino acid sequence identity to SEQ ID NO:4.

By an amino acid sequence having x% identity to a reference sequence such SEQ ID NO:4, is meant a sequence which is identical to the reference sequence except that it may include up to 100-x amino acid alterations per each 100 amino acids of the reference sequence. For example, in a subject amino acid sequence having at least 80% identity to a reference sequence, up to 20% of the amino acid residues in the reference sequence may be substituted, deleted or inserted with another amino acid residue. Percentage identity between amino acid sequences can be determined conventionally using known computer programs, for example the FASTDB program based on the algorithm of Brutlag et al (Comp.App.Biosci. (1990) 6:237-245).

The isolated polynucleotide of the invention may be cDNA, genomic DNA or RNA. In particular embodiments, the isolated polynucleotide is cDNA comprising the nucleotide sequence of SEQ ID NO:1 or SEQ ID NO:2 or SEQ ID NO:3 or SEQ ID NO:5, a genomic DNA comprising the nucleotide sequence of SEQ ID NO:6 or SEQ ID NO:7 or a DNA comprising a nucleotide sequence which hybridises to SEQ ID NO:1 or SEQ ID NO:2 or SEQ ID NO:3 or SEQ ID NO:5 or SEQ ID NO:6 or SEQ ID NO:7 under stringent conditions.

The invention also provides an isolated polynucleotide comprising a consecutive 20 base pair nucleotide portion identical in sequence to a consecutive 20 base pair portion of SEQ ID NO:1 or SEQ ID NO:2 or SEQ ID NO:3 or SEQ ID NO:5 or SEQ ID NO:6 or SEQ ID NO:7.

A polynucleotide of the invention may be isolated by bioinformatics analysis of DNA sequences from the subregion 5q31-5q33 on chromosome 5 determined by sequencing of yeast artificial chromosomes (YACs), bacterial artificial chromosomes (BACs) and/or P1 artificial chromosomes (PACs) to identify genes within that subregion, searching for a sequence having greater than 95% identity to the predicted exon for a selected gene and isolating cDNA from a human fetal brain, adult brain, kidney, lung, muscle, peripheral blood lymphocyte, small intestine, spleen or testis cDNA library by PCR using primers designed using that sequence.

A polynucleotide of the invention, for example having the SEQ ID NO:1 or SEQ ID NO:2 or SEQ ID NO:3 or SEQ ID NO:5 or SEQ ID NO:6 or SEQ ID NO:7 may be prepared from the nucleotides which it comprises by chemical synthesis, e.g. automated solid phase synthesis using known procedures and apparatus.

In another aspect, the present invention provides an isolated polypeptide, particularly a recombinant polypeptide, comprising the amino acid sequence of SEQ ID NO:4 or a functionally equivalent variant thereof. Such a polypeptide may be produced by cloning a polynucleotide sequence as hereinbefore described into an expression vector containing a promoter and other appropriate regulating elements for transcription, transferring into prokaryotic or eukaryotic host cells such as bacterial, plant, insect, yeast, animal or human cells, and culturing the host cells containing the recombinant expression vector under suitable conditions. Techniques for such recombinant expression of polypeptides are well known and are described, for example, in J. Sambrook et al, Molecular Cloning, second edition, Cold Spring Harbor Press, 1990.

Accordingly, the present invention also provides a method of producing a polypeptide of the invention which comprises culturing a host cell containing an expression vector containing a polynucleotide sequence of the invention as hereinbefore described under conditions suitable for expression of the polypeptide and recovering the polypeptide from the host cell culture.

In another aspect, the present invention provides an expression vector containing a polynucleotide sequence of the invention as hereinbefore described.

The invention also provides an isolated polypeptide comprising a consecutive 10 amino acid portion identical in sequence to a consecutive 10 amino acid portion of SEQ ID NO:4.

A polypeptide of the invention may be expressed as a recombinant fusion protein with one or more heterologous polypeptides, for example to facilitate purification. For example, it may be expressed as a recombinant fusion protein with a heterologous polypeptide such as a polyhistidine containing a cleavage site located between the polynucleotide sequence of the invention and the heterologous polypeptide sequence, so that the polypeptide comprising the amino acid sequence of SEQ ID NO:4 may be cleaved and purified away from the heterologous moiety using well known techniques.

A polypeptide of the invention may also be synthesised, in whole or in part, from the amino acids which it comprises using well known chemical methods, for example automated solid phase techniques.

Isolated polypeptides of the invention as hereinbefore described may be purified by well known standard procedures.

The present invention also provides a variant of a polynucleotide of the invention as hereinbefore described, particularly a polynucleotide having a SEQ ID NO:1 or SEQ ID NO:2 or SEQ ID NO:3 or SEQ ID NO:5 or SEQ ID NO:6 or SEQ ID NO:7, which contains a sequence polymorphism correlated with a disease, particularly asthma. The polymorphism may be an addition, deletion or replacement of one or more nucleotides. Single nucleotide polymorphisms (SNPs), as compared with SEQ ID NO:1 or SEQ ID NO:2 or SEQ ID NO:3 or SEQ ID NO:5 or SEQ ID NO:6 or SEQ ID NO:7, which have been identified in genetic samples from asthmatic patients are shown hereinafter in the Examples.

The present invention further provides a variant of the polypeptide comprising the amino acid sequence of SEQ ID NO:4, which variant (hereinafter described alternatively as mutant protein) is an isolated polypeptide which is encoded by a variant of a nucleotide sequence of a polynucleotide of the invention as hereinbefore described, particularly a variant of SEQ ID NO:1 or SEQ ID NO:2 or SEQ ID NO:3 or SEQ ID NO:5 or SEQ ID NO:6 or SEQ ID NO:7, having a sequence polymorphism correlated with a disease, particularly asthma. Such mutant proteins can be expressed or synthesised analogously to the polypeptide comprising SEQ ID NO:4. The present invention also provides an antibody which is immunoreactive with a polypeptide of the invention as hereinbefore described, or a mutant protein as hereinbefore described. The antibody may be a polyclonal or monoclonal antibody. Such antibodies may be prepared using conventional procedures. Methods for the production of polyclonal antibodies against purified antigen are well established (cf. Cooper and Paterson in Current Protocols in Molecular Biology, Ausubel et al. Eds., John Wiley and Sons Inc., Chapter 11). Typically, a host animal, such as a rabbit, or a mouse, is immunised with a purified polypeptide or mutant protein of the invention, or immunogenic portion thereof, as antigen and, following an appropriate time interval, the host serum is collected and tested for antibodies specific against the polypeptide. Methods for the production of monoclonal antibodies against purified antigen are well established (cf. Chapter 11, Current Protocols in Molecular Biology, Ausubel et al. Eds., John Wiley and Sons Inc.). For the production of a polyclonal antibody, the serum can be treated with saturated ammonium sulphate or DEAE Sephadex. For the production of a monoclonal antibody, the spleen or lymphocytes of the immunised animal are removed and immortalised or used to produce hybridomas by known methods. Antibodies secreted by the immortalised cells are screened to determine the clones which secrete antibodies of the desired specificity, for example using Western blot analysis. Humanised antibodies can be prepared by conventional procedures.

In another aspect, the present invention provides an antisense oligonucleotide comprising a nucleotide sequence complementary to that of a polynucleotide of the invention or a variant thereof having a polymorphism correlated with a disease, particularly asthma, in particular a nucleotide sequence complementary to SEQ ID NO:1 or SEQ ID NO:2 or SEQ ID NO:3 or SEQ ID NO:5 or SEQ ID NO:6 or SEQ ID NO:7, or a polymorphic variant thereof, The antisense oligonucleotide may be DNA, an analogue of DNA such as a phosphorothioate or methylphosphonate analogue of DNA, RNA, an analogue of RNA, or a peptide nucleic acid (PNA). The antisense oligonucleotides may be synthesised by conventional methods, for example using automated solid phase techniques.

The present invention also provides a polynucleotide probe comprising at least 15 contiguous nucleotides of a polynucleotide of the invention or a complement thereof. The probe may be cDNA, genomic DNA or RNA. Usually it is a synthetic oligonucleotide comprising 15 to 50 nucleotides, which can be labelled, e.g. with a fluorophore, to provide a detectable signal.

The polynucleotide probe is capable of selectively hybridising under stringent conditions to a polynucleotide fragment having a sequence selected from the group consisting of SEQ ID NOs: 1, 2, 3, 5, 6 and 7. The probe has a sequence such that under such hybridisation conditions it hybridizes only to its cognate sequence. DNA probes as described above are useful in a number of screening applications including Northern and Southern blot analyses, dot blot and slot blot analyses, and fluorescence in situ hybridisation (FISH).

The present invention also includes a pair of oligonucleotides having nucleotide sequences useful as primers for DNA amplification of a fragment of a polynucleotide of the invention, i.e. of the human AAGB gene (hAAGB), wherein each primer of said pair is at least 15 nucleotides in length and said pair have sequences such that when used in a polymerase chain reaction (PCR) with either human genomic DNA or a suitable human cDNA target they result in synthesis of a DNA fragment containing all or preferably part of the sequence of hAAGB. The primer pair is preferably capable of amplifying at least one exon of hAAGB (or portion thereof), such as an exon selected from those in SEQ ID NO:1, 2, 3, 5, 6 or 7. Examples of such primer pairs are shown hereinafter in the Examples. Exemplary applications of such primer pairs include amplification of DNA fragments for use in the detection of changes to the polynucleotide sequence in asthmatic patients as shown hereinafter in the Examples.

The role of the polypeptide of the invention in asthma and other obstructive or inflammatory airways diseases characterised by bronchial hyperresponsiveness can be determined using conventional allergen driven animal models for bronchial hyperresponsiveness, e.g. the ovalbumin-induced BHR mouse model (Tsuyuki et al, J. Clin. Invest. 96:2924-2931) or the guinea pig model hereinafter described.

Polynucleotides, polypeptides, antibodies, antisense oligonucleotides or probes of the invention as hereinbefore described, hereinafter alternatively referred to collectively as agents of the invention, may be used in the treatment (prophylactic or symptomatic) or diagnosis of inflammatory or obstructive airways diseases. For example, a polypeptide of the invention may be used to treat a mammal, particularly a human, deficient in or otherwise in need of that polypeptide; a polynucleotide of the invention may be used in gene therapy where it is desired to increase AAGB activity, for instance where a subject has a mutated or missing AAGB gene; an antisense oligonucleotide of the invention may be used to inhibit AAGB activity or activity of variants of the AAGB gene having a polymorphism correlated with a disease, e.g. asthma, where this is desired; an antibody of the invention may be used to detect, or determine the level of expression of, AAGB polypeptides or a disease-correlated polymorphic variant thereof, or to inhibit ligand/antiligand binding activities of AAGB polypeptides; and a probe of the invention may be used to detect the presence or absence of the AAGB gene, i.e. to detect genetic abnormality.

“Gene therapy” refers to an approach to the treatment of human disease based upon the transfer of genetic material into somatic cells of an individual. Gene transfer can be achieved directly in vivo by administration of gene-bearing viral or non-viral vectors into blood or tissues, or indirectly ex vivo through the introduction of genetic material into cells manipulated in the laboratory followed by delivery of the gene-containing cells back to the individual. By altering the genetic material within a cell, gene therapy may correct underlying disease pathophysiology. Suitable vectors, and procedures, for gene delivery to specific tissues and organ systems in animals are described in Dracopoli, N. C. et al., Current Protocols in Human Genetics. John Wiley and Sons Inc., Chapters 12 and 13 respectively. In relation to polynucleotides of the invention, gene therapy may involve delivery of a viral or non-viral gene therapy vector containing an expression cassette of the AAGB gene under suitable control elements to the lungs of diseased individuals (eg. asthmatics) so that the underlying disease pathophysiology is corrected or ameliorated.

Accordingly, in further aspects, the present invention provides

a pharmaceutical composition comprising a polynucleotide, polypeptide, antibody or antisense oligonucleotide of the invention as hereinbefore described, optionally together with a pharmaceutically acceptable carrier;

a method of treating an inflammatory or obstructive airways disease which comprises administering to a subject in need thereof an effective amount of a polynucleotide, polypeptide, antibody or antisense oligonucleotide of the invention as hereinbefore described;

a method of detecting genetic abnormality in a subject which comprises incubating a genetic sample from the subject with a polynucleotide probe of the invention as hereinbefore defined, under conditions where the probe hybridises to complementary polynucleotide sequence, to produce a first reaction product, and comparing the first reaction product to a control reaction product obtained with a normal genetic sample, where a difference between the first reaction product and the control reaction product indicates a genetic abnormality in the subject or a predisposition to developing a disease such as asthma;

a method of detecting the presence of a polynucleotide of the invention, e.g. comprising SEQ ID NO:1, 2, 3, 5, 6 or 7, in cells or tissues which comprises contacting DNA from the cell or tissue with a polynucleotide probe as hereinbefore defined under conditions where the probe is specifically hybridizable with a polynucleotide of the invention, and detecting whether hybridization occurs;

a method of detecting an abnormality in the nucleotide sequence of a polynucleotide of the invention in a patient which comprises amplifying a target nucleotide sequence in DNA isolated from the patient by a polymerase chain reaction using a pair of primers as hereinbefore described which target the sequence to be amplified and analysing the amplified sequence to determine any polymorphism present therein; and

a method of detecting polymorphism in a subject which comprises treating a tissue sample from the subject with an antibody to a mutant protein of the invention and detecting binding of said antibody.

The term “polymorphism” means any sequence difference as compared with the sequence of a polynucleotide of the invention as hereinbefore described.

Hybridisation of a polynucleotide probe of the invention with complementary polynucleotide sequence may be detected using in situ (eg. FISH) hybridization, Northern or Southern blot analyses, dot blot or slot blot analyses. The abnormality may also be detected for example by conformation sensitive gel electrophoresis (CSGE) and DNA sequencing as described hereinafter in the Examples. The genetic abnormality may result in a change in the amino acid sequence of the individual's AAGB protein relative to the the amino acid sequence of a normal hAAGB protein, or loss of protein. Alternatively, the change may not alter the amino acid sequence but may instead alter expression of the AAGB gene by altering the sequence of controlling elements either at the 5′-, or 3′-end of the gene, or altering the sequence of control elements within intronic regions of the gene. Changes may also affect the way the gene transcript is processed or translated. The invention also includes kits for the detection of an abnormality in the polynucleotide sequence of an individual's AAGB gene. Hybridisation kits for such detection comprise a probe of the invention as hereinbefore described, which probe may be modified by incorporation of a detectable, e.g. chemiluminescent or fluorescent, label therein, and may include other reagents such as labelling reagents, i.e. reagents to incorporate a detectable label such as a radioactive isotope, chemiluminescent or fluorescent group into a hybridised product, and buffers. PCR amplification kits comprise primer pairs such as those described above together with a DNA polymerase such as Taq polymerase, and may include additional reagents, such as an amplification buffer and the like. Specific embodiments of the PCR amplification kits can include additional reagents specific for a number of techniques that detect polynucleotide changes, including CSGE and DNA sequencing.

The effectiveness of an agent of the invention in inhibiting or reversing airways hyperreactivity may be demonstrated in a guinea pig test model. The acute injection of pre-formed immune complex renders guinea pigs hyperreactive to histamine. Doses of histamine which cause only a small degree of bronchoconstriction prior to administration of immune complex cause a much stronger effect thereafter. Guinea-pigs (Dunkin-Hartley, male, 400-600 g) are anaesthetised with phenobarbital (100 mg/kg i.p.) and pentobarbital (30 mg/kg i.p.) and paralysed with gallamine (10 mg/kg i.m.) and ventilated with a mixture of air and oxygen (45:55), v/v). Animals are ventilated (8 ml/kg, 1 Hz) via a tracheal cannula. Ventilation is monitored by a flow transducer. When making measurements of flow, coincident pressure changes in the thorax are monitored directly via an intrathoracic trochar, permitting display of differential pressure relative to the trachea. From this information resistance and compliance are calculated at each inspiration. An allergic reaction is initiated by intravenous injection of preformed immune complexes (prepared by adding 30 μg of bovine gamma globulin in 0.05 ml of saline to 0.05 ml of guinea pig anti-bovine gamma globulin anti-serum) 3 times at 10 minute intervals. Intravenous injections of histamine (1.0-3.2 μg/kg at 10 minute intervals) are used to define the sensitivity of the airways prior to and following the last exposure to the immune complex. Airways hyperreactivity is expressed as the paired difference for the maximal value of lung resistance in response to histamine before and after repeated injection of immune-complex. The agents of the invention are administered intratracheally either as solutions or suspensions in tragacanth. The ED₅₀₋ values for reversal of airways hyperreactivity are determined graphically from the dose response curves and represent those doses which cause a 50% reduction of airways hyperreactivity.

Inflammatory or obstructive airways diseases to which the present invention is applicable include asthma of whatever type or genesis including both intrinsic (non-allergic) asthma and extrinsic (allergic) asthma. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or reduced airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include adult respiratory distress syndrome (ARDS), chronic obstructive pulmonary or airways disease (COPD or COAD), including chronic bronchitis, or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, agents of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

The agents of the invention may be administered by any appropriate route, e.g. orally, for example in the form of a tablet or capsule; parenterally, for example intravenously; topically, e.g. in an ointment or cream; transdermally, e.g. in a patch; by inhalation; or intranasally.

Pharmaceutical compositions containing agents of the invention may be prepared using conventional diluents or excipients and techniques known in the galenic art. Thus oral dosage forms may include tablets and capsules, and compositions for inhalation may comprise aerosol or other atomizable formulations or dry powder formulations.

The invention includes (A) an agent of the invention in inhalable form, e.g. in an aerosol or other atomizable composition or in inhalable particulate, e.g. micronised form, (B) an inhalable medicament comprising an agent of the invention in inhalable form; (C) a pharmaceutical product comprising such an agent of the invention in inhalable form in association with an inhalation device; and (D) an inhalation device containing an agent of the invention in inhalable form.

Dosages of agents of the invention employed in practising the present invention will of course vary depending, for example, on the particular condition to be treated, the effect desired and the mode of administration. In general, suitable daily dosages for administration by inhalation are of the order of 1 μg to 10 mg/kg while for oral administration suitable daily doses are of the order of 0.1 mg to 1000 mg/kg.

The present invention further provides a method of determining predisposition of a subject to asthma comprising determining the presence or absence in DNA from the subject of a sequence polymorphism in a polynucleotide of the invention which correlates with asthma.

In another aspect, the present invention provides a method of determining predisposition of a patient to asthma which comprises identifying in DNA from the patient a sequence polymorphism or haplotype in a polynucleotide of the invention, as compared with a normal control DNA from a non-asthmatic subject, which correlates with asthma. A haplotype is a set of polymorphisms which is inherited together as a group.

In a related aspect, the invention provides a method of determining predisposition of a patient to asthma which comprises identifying in DNA from the patient a sequence polymorphism or haplotype in a polynucleotide of the invention, as compared with SEQ ID NO:1 or SEQ ID NO:2 or SEQ ID NO:3 or SEQ ID NO:5 or SEQ ID NO:6 or SEQ ID NO:7, which correlates with asthma.

Identification of a sequence polymorphism may be effected by conventional sequencing and sequence analysis techniques, for example as described in Cotton, R. G. H., Mutation Detection, Oxford University Press, 1997; Landegren, U., Laboratory Protocols for Mutation Detection, Oxford University Press; and R. G. H. Cotton et al, Mutation Detection, Oxford University Press, 1998.

Sequence polymorphisms which correlate with asthma may alter the amino acid sequence in the encoded polypeptide or may affect expression levels of the polypeptide or the way in which a transcript is processed.

Certain sequence polymorphisms or haplotypes may correlate with the severity and/or nature of the asthmatic phenotype, e.g. with mild, moderate or severe asthma as defined by established clinical parameters. Identification of polymorphisms may therefore be useful for prognosis, determination of therapeutic strategy and prediction of patient responses to therapy.

A polypeptide or mutant protein of the invention can be used to identify enhancers (agonists) or inhibitors (antagonists) of its activity, i.e. to identify compounds useful in the treatment of inflammatory or obstructive airways diseases, particularly asthma. Accordingly, the invention also provides a method of identifying a substance which modulates the activity of a polypeptide of the invention comprising combining a candidate substance with a polypeptide or mutant protein of the invention and measuring the effect of the candidate substance on said activity. The activity of a polypeptide or mutant protein of the invention may be measured, for example, by measuring changes in levels of intracellular components such as Ca²⁺ or cAMP, by a shape change assay or by an appropriate reporter gene assay. The invention also includes a method of identifying a substance which binds to a polypeptide or mutant protein of the invention comprising mixing a candidate substance with a polypeptide or mutant protein of the invention and determining whether binding has occurred.

The invention is illustrated by the following Examples, with reference to the accompanying drawings.

Abbreviations used in the Examples have the following meanings:

| | |
|---|---|
| AEBSF: | 4-(2-Aminoethyl)benzenesulfonyl fluoride |
| BAC: | bacterial artificial chromosome |
| BAP: | 1,4-bis(acryloyl)piperazineBLAST: basic local alignment |
| | search tool |
| BSA: | bovine serum albumin |
| CSGE: | conformation sensitive gel electrophoresis |
| DNTP | deoxynucleotide triphosphate |
| DTT: | dithiothreitol |
| EGF: | epidermal growth factor |
| EIA: | enzyme immunoassay |
| EST: | expressed sequence tag |
| FCS: | fetal calf serum |
| GPCR: | G-protein coupled receptor |
| MTN: | multiple tissue northern |
| NCBI: | National Center for Biotechnology Information |
| ORF: | open reading frame |
| PAC: | P1 artificial chromosome |
| PBS: | phosphate buffered saline |
| PBL: | peripheral blood lymphocyte |
| PEG: | polyethylene glycol |
| PMSF: | Phenylmethylsulfonyl fluoride |
| SDS-PAGE: | sodium dodecyl sulfate polyacrylamide gel electrophoresis |
| SNP | single nucleotide polymorphism |
| STS | sequence tagged site |
| TGF: | transforming growth factor |
| TTE: | 44 mM Tris, 14.5 mM taurine, 0.1 mM EDTA, pH 9.0 |

### EXAMPLE 1

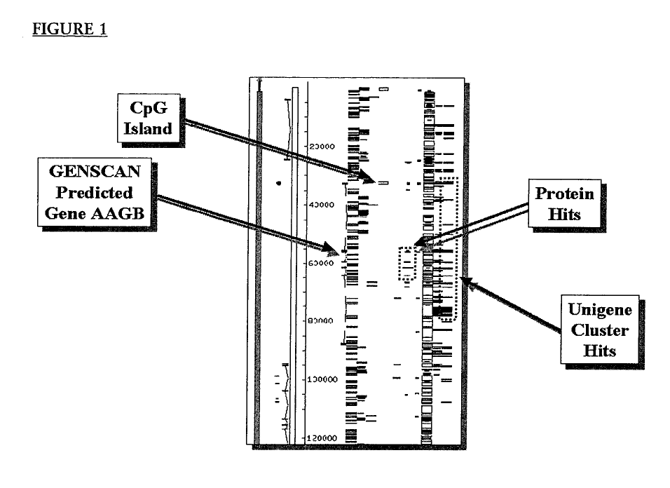

Bacterial artificial chromosome (BAC) clones identified using physical map information for human chromosome 5q31-q33 publicly available on the Lawrence Berkley National Laboratory Genome Centre web site (LBNL; www-hgc.lbl.gov/biology/bacmap/2.gif) obtained as BAC clone numbers h164 (22f14), c5 (50g20), h187 (35k5), h167 (8e5) and h177 (32d16) from Research Genetics (Huntsville, Ala., USA), and a P1 artificial chromosome (PAC) isolated by PCR using primers with SEQ ID NOS:11 to 14 for the STS markers bac51107T (5′ end of BAC 50g20) and bac51330T (3′ end of BAC 22f14) available on the LBNL website (www₁₃hgc.lbl.gov/sts.html) by Genome Systems Inc. (St. Louis, Mo., USA), the BACs and PAC together covering a sub-region of human chromosonal region 5q31-5q33, are sequenced using conventional techniques for an ABI 377 sequence (http://www.pebio.com/ab/about/dna/377/). The resulting genomic DNA sequence is analysed using SCAN (Burge and Karlin, J. Mol. Biol. 268:78-94) and GENEMARK version 2.4 (Borodovsky and McIninch, Comp. Chem. 17:123-133) gene-finding programs and BLAST (Altschul et al., J. Mol. Biol. 215:403-410) homology searches against public protein, EST and DNA databases (SWISSPROT, SWISSPROTPLUS, GenBank, Genbank updates, EMBL, GENEMBLPLUS, GenBank EST, EMBL EST, GenBank STS, EMBL STS), the results of which are parsed into a human chromosome 5-specific version of ACeDb (A C. elegans Database; http://www.sanger.ac.uk/Software/Acedb/) for graphic display. From this graphic display, shown in FIG. 1, significant regions (i.e. genes) are identified by predicted exons and alignedEST/protein hits. A gene AAGB is initially identified on the graphic display as a GENSCAN-predicted gene covering 55 kb of genomic DNA and comprising 9 exons ranging in size from 31-132 bp:

| | | |
|---|---|---|
| GENSCAN- | Nucleotide Position In: | Exon Size |

| Predicted Exon | SEQ ID No. 6 | SEQ ID No. 7 | (bp) |
|---|---|---|---|
| 1 | 1212-1274 | — | 63 |
| 2 | 1343-1474 | — | 132 |
| 3 | 24053-24134 | — | 82 |
| 4 | 24451-24581 | — | 131 |
| 5 | 27969-28056 | — | 88 |
| 6 | 29972-30096 | — | 125 |
| 7 | 32801-32867 | — | 67 |
| 8 | — | 6209-6239 | 31 |
| 9 | — | 6486-6564 | 79 |

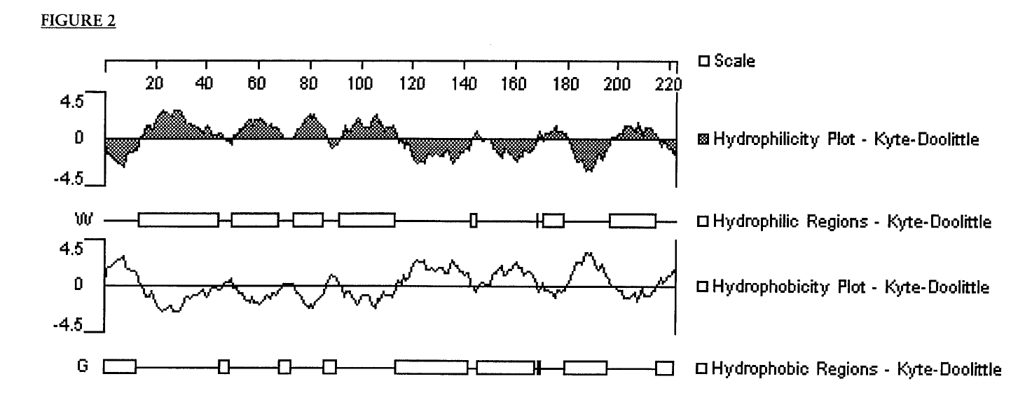

A number of hits are found in the EST database. From these EST hits a Unigene cluster (Hs.9788) is identified. Assembly of the ESTs in this cluster using the SeqMan module of LAsergene software (DNASTAR, Inc., Madison, Wis., USA) produces 2 contigs of ESTs of 2057 bp (SEQ ID NO:1) and 725 bp (SEQ ID NO:2). PCR primers having SEQ ID NOS: 8, 9 and 10 were used to screen cDNA libraries from fetal brain, adult brain, kidney, lung, muscle, PBL, small intesrine, spleen and testis. All give a positive result on screening, but the longest clone is found in the small intestine library. The cDNA insert is sequenced using primer-directed walking. The resulting 1054 base pair of insert sequence (SEQ ID NO:5) aligns with sequence SEQ ID NO:1 at position 859-1893, therefore, the cDNA sequence of SEQ ID NO:5 does not extend the Unigene cluster sequence, or bridge the sequence gap between SEQ ID NO:1 and SEQ ID NO:2. Using the reverse complement of SEQ ID NO:2 positions 725-605, a 3599 bp EST clone (232376.4; SEQ ID NO:3) is identified by BLAST screening of the LifeSeq® Gold commerical database (Incyte Pharmaceuticals Inc., 3160 Porter Drive, Palo Alto, Calif. 94304, USA). Alignment of SEQ ID NOS:1, 2 and 3 shows that clone 232376.4 contains the DNA sequences represented by SEQ ID NOS:1 and 2 and that the DNA sequence between positions 1942 and 2887 of SEQ ID NO:3 bridges the 946 bp gap between SEQ ID NOS: 1 and 2. Alignment of SEQ ID NO: 3 with the genomic sequence (SEQ ID NO:6) reveals 8 exons spread over 45 kb of genomic DNA. The genomic structure of the AAGB gene and its to SEQ ID NOS: 1, 2, 3, 5 and 6 are shown in FIG. 3.

| | | Nucleotide Position | |
|---|---|---|---|
| Exon | SEQ ID NO: 3 | in SEQ ID NO: 6 | Exon Size (bp) |
| 1 | 48-285* | 1037-1274 | 238 |
| 2 | 286-373* | 24047-24132 | 88 |
| 3 | 374-505* | 24451-24582 | 132 |
| 4 | 506-592* | 27970-28056 | 87 |
| 5 | 593-717* | 29972-30096 | 125 |
| 6 | 718-785* | 32801-32868 | 68 |
| 7 | 786-890 | 36815-36920 | 106 |
| 8 | 891-3599 | 43976-46684 | 2708 |

*match with GENSCAN-predicted exon.

Analysis of SEQ ID NO:3 using the EditSeq module of the Lasergene software (DNASTAR, Inc., Madison, Wis., USA) reveals that the longest open reading frame (ORF) encodes a protein of 221 amino acids (SEQ ID NO:4; encoded by nucleotides 223-888 of SEQ ID NO: 3). Analysis of this putative protein using the Kyte-Doolittle algorithm (Kyte and Doolittle J. Mol. Biol. 157:105-132) in the Protean module of the Lasergene software indicates the presence of 4 hydrophilic domains in the N-terminal half of the molecule and 3 hydrophobic domains in the C-terminal half, indicating that the protein is a transmembrane protein (FIG. 2).

Further analysis on the SOSUI system (http://azusa.proteome.bio.tuat.ac.jp/sosui/) identifies 3 transmembrane helices corresponding to the 3 predicted hydrophobic domains:

| | N | | C | |
|---|---|---|---|---|
| No. | Terminal | Transmembrane Region | Terminal | Length |
| 1 | 117 | IFMLTFFMAFLFNWIGFFLSFCL | 139 | 23 |
| 2 | 148 | GAISGFGLSLIKWILIVRFSTYF | 170 | 23 |
| 3 | 179 | WLWWVFLVLGFLLFLRGFINYAK | 201 | 23 |

Further analysis of the three transmembrane domains by searches against the MOTIF database (www.motif.genome.ad.jp) reveals hits for all three domains against the rhodopsin-like GPCR superfamily signature [Attwood and Findlay, Prot. Engng. 7:195-203 and 6: 167-176]. The presence of only three hydrophobic membrane-spanning regions, rather than seven (typical signature of a GPCR), indicates that the protein encoded by 232376.4 is not a member of the GPCR family, but is a member of an alternative family of transmembrane proteins.

BLASTP searching of the NR protein database using SEQ ID NO:4 as the query sequence identifies significant hits with protein sequences AK001723 (putative protein translation of a human cDNA derived from a teratocarcinoma cell line), AB032991 (KIAA1165 protein derived from a cDNA clone derived from an adult male brain cDNA library) and AAF49316 (CG8056 gene product; a predicted protein from the Drosophila sequencing effort). Alignment of SEQ ID NO: 4 with the AK001723, AB032991 and AAF49316 protein sequences using Clustal 1.7 (Thompson, Higgins and Gibson Nucleic Acids Res. 22:4673-4680) shows that the three predicted transmembrane domains in 232376.4 are conserved in the three protein database hits (FIG. 4).

Moreover, the alignment shows that AB032991 and AK001273 are identical, except that the former is 40 amino acids longer. The gene encoding AB032991/KIAA1165 is expressed in a number of tissues and has been assigned to chromosome 13 by radiation hybrid mapping (http://www.kasuza.or.jp/huge). Examination of the FlyBase (http://flybase.bio.indiana.edu:7081/) database for further details of the AAF49316/CG8056 protein (Fban0008056) reveals that the protein is encoded by the Drosophila Keren gene. The Keren gene encodes two putative proteins of 217 amino acids (alt1; AAF49315) and 267 amino acids (alt 2; AAF49316). Alt 1 and alt 2 do not display any homology with each other and only alt 2 has homology with SEQ ID NO: 4. All protein sequences similar to alt 1 and alt 2 in the NR database are accessed using the Drosophila precomputed BLAST facility at the NCBI (http://www.ncbi.nlm.nih.gov/PMGifs/Genomes/7227.html). Alt 1 has homologies with 223 proteins in a range of organisms, many of which are transmembrane proteins containing EGF motifs. In contrast, alt 2 has a proline rich motif and displays homologies to only 2 other proteins: AB032991/KIAA1165 and AAF11803 (a putative erythomycin esterase from Deinococcus radiodurans).

Taken together, the Protean, SOSUI and BLAST analyses indicate that the AAGB gene encodes a novel transmembrane protein.

Using the cDNA clone insert corresponding to SEQ ID NO:5, a northern blot of mRNA from a number of human tissues (human 12-lane MTN blot; Clontech Laboratories UK Ltd., Basingstoke, Hampshire, UK) is probed to examine the expression profile of gene AAGB. Two hybridising bands of ˜4.0 kb and 2.0 kb are detected which are most strongly visulaised in brain, heart, kidney and liver RNA and at lower levels in skeletal muscle, spleen, placenta and lung. Very low levels are found in colon, thymus, small intestine and peripheral blood lympocytes. PCR analysis of first-strand cDNAs derived from 24 different tissues (Rapid-Scan™ Gene Expression Panel; OriGene Technologies Inc., Rockville, Md., USA) using primers having SEQ ID NOS:15 and 18 confirms the Northern blot results.

### EXAMPLE 2

In this example conformation sensitive gel electrophoresis (CSGE: Ganguly et al., Proc. Natl. Acad. Sci. USA 90:10325-10329; Ganguly and Williams, Hum. Mut. 9:339-343) is used to detect potential sequence changes in PCR-amplified DNA fragments from blood DNA isolated from asthmatic patients. Single base mismatches in DNA heteroduplexes are detected by polyacrylamide gel electrophoresis in the presence of mildly denaturing solvents which amplify the tendency of mismatches to produce conformational changes and result in differential migration of homo-duplexes and heteroduplexes. To generate heteroduplexes, amplified PCR products are thermally denatured, annealed, then analysed by polyacrylamide gel electrophoresis. DNA fragments are visualised by ethidium bromide staining. DNA fragments showing differential electrophoretic migration patterns are then sequenced to confirm the presence of a change to the polynucleotide sequence and the exact nature of this change.

PCR primer sets covering the 8 exons (including the exon-intron boundaries) identified by alignment of SEQ ID NO:3 with SEQ ID NO: 6 (Example 1), the first 1 kb of intron 1 and 1 kb of the 3′-end of the AAGB gene are designed using SEQ ID NO:6 and Primer Express™ (version 1.0; Perkin Elmer, P/N 604313). These primer sets (SEQ ID NOs: 18-84) are:

| Alias | Forward | Reverse |
|---|---|---|
| Prom 1.1 | CTTCACCCCYIAGTAGCTCTGTGTC | AATCCTTGAGAACTGCTTGGATCT |
| Prom 1.2 | CCCTTTGCACCTGTTCCTGT | TCCGGTCTGGCACCATG |
| Prom 1.3 | AAATGTCAGATAGCAAGTATTATAGGGTTTG | TGCCAAGCACTGAGCTTCAC |
| Prom 1.4 | AACCACGATTCTGCTGCCA | GCCCCACCCGCGACT |
| Prom 1.5 | ACACTTGAGCCTCGGAGGC | CAGGGAATCAGAGCGAGCG |
| Exon 1 | CCCGCCTCTTCCCCAG | AGCCCCGGCGTCGA |
| Intron 1.1 | TGTCGACTGGGCCATGG | CCGCCTGTGGGTCGC |
| Intron 1.2 | GCCTCAGGCCTCTCTGGC | GAGCTATGGGCAGCAGCAG |
| Intron 1.3 | TCCCTGAGTCAGAACCCGC | AATACACCCGGCACACGC |
| Intron 1.4 | CCCCTCGGCTCCGTTTT | CCACCATTACTGTATGTTAGATTAACACTTC |
| Intron 1.5 | TCATTTTCTCGGGTTTGATTTAATACA | AAACCAGTAACCGGCCG |
| Exon 2 | TCCATAGAATAACTTCCTGGGAGTAGAAT | TTGTAACCTTTCTTCCAACTGCTTC |
| Exon 3 | TGGTTCAATTGTTATGTATGTACTCTTAAGG | AAAATTAGCTCTACTTTCACTAATCAATACAAT |
| Exon 4 | GAATTTTAAACACACATTGAGCCTTTAAT | CCAGTAAAAGGCACCAAATAAACATA |
| Exon 5 | GGCAGGTGTAAGGCACTCTTG | TGTGCATGAACCATTTGCAA |
| Exon 6 | GCATTTTGTGATTTTGTGTACCCTT | TTAGGAAGAACCTACAACCACAAAGA |
| Exon 7 | CAGATTCTGGAGGAAATTGCTAATGT | GGGACTGTGCCTTCTTTACTTTCTCT |
| Exon 8.a | TTGTCTTAGCCAAAATAACAAGCTAACTAGA | GGACATGGCTCATGCTACTTAAAATG |
| Exon 8.b | GATCATTTCTCTCTATTTGTTCCTAGGTGT | CATACAGAATTGAATAAATTTCTAGATTAACTTGAA |
| Exon 8.c | CATGTCCCTGTAGTCGGTAGGG | GTTTTATACAGTGACAACACCTTTAGAATCC |
| Exon 8.d | AACACCTGGAAGCAAAATCATAGTG | GGATCATCTCAAAAGTTCCAAGCTACT |
| Exon 8.e | AATTCTTGACACTCTTTCTATAATTAGCGTTC | ATCAACAAATGCAAGCCAAGAAAT |
| Exon 8.f | AATGTGTTAGCAGAAACCAGTGGG | CTCTACAACTCCTCTCTACTTTTTTAGTCCAT |
| Exon 8.g | TTGTATGGCCTATAATGTTCTAAGTTCCA | CCACTTCATGTGCCTCAAAAGATT |
| Exon 8.h | CGCCTTATTTGGTTTTGTATATTCAAC | CGCGGTTCCTATGAATACCTTTC |
| Exon 8.i | GAGTCCTTACTGGTATTCAGGATGAAA | CAAGCAAATTTTAATAATGCCTTTTATTTC |
| Exon 8.j | GTTATGGAGTAAACTAGCTTGGACCTTG | TTCATCTGTACTAATAATGACTCACGGTG |
| 3′.1 | GCTGGAATGCAGTGTTGCG | TTGAAATATCACTTCTATTGGTCCTGA |
| 3′.2 | GACCTCGTGATCCGCCC | ACAAGAAGCACGGTCAAATGATT |
| 3′.3 | AATTTGAGTGATTGCCTGAGGG | GCCAAGATCACGACTTTGTACTTAAGT |
| 3′.4 | ACAAAGTCGTGATCTTGGCTCA | GCTGAACCACCCACAGAACCT |
| 3′.5 | GTTGTGACCCCTATTACCTATGTGAAT | CACAAACGTGTACAGGTATACGCAA |
| 3′.6 | GGTGGATTGGGTGAGAGCATT | CTATAGGCCATAAAGTTGTAGGTGATACA |

Using the above primer sets, 33 polynucleotides are amplified from blood DNA samples from 16 asthmatic patients. PCR reactions are carried out in a reaction volume of 10 μl containing 1×GeneAmpe® 10×PCR buffer (Perkin Elmer P/N N808-0240), 13 ng of template DNA, 400 μM of each dNTP (Amersham Life Science Nucleix Plus™ 25 mM dNTP mix; Prod. No. US77119), 30 ng of each primer, 2 mM MgCl₂ and 0.5 u of AmpliTaq Gold™ polymerase (Perkin-Elmer P/N N808-0242).

Typical thermal cycling conditions using a Biometra UNO II cycler (Part No. 050-603; Anachem Ltd., Luton, UK) are as follows, the sequence Step 2-Step 3-Step 4 being repeated 36 times:

| | | |
|---|---|---|
| Step 1 | 95° C. | 10 min |
| Step 2 | 92° C. | 1 min |
| Step 3 | 60° C. | 1 min |
| Step 4 | 72° C. | 2 min |
| Step 5 | 72° C. | 10 min |

To generate heteroduplexes, 2 μl of PCR product is denatured at 95° C. for 10 minutes and annealed at 68° C. for 30 minutes using a thermal cycler (eg. Biometra UNOII). 2 μl of 2×loading buffer (20% ethylene glycol, 30% formamide, 0.025% xylene cyanol, 0.025% bromphenol blue) is added to each sample before gel analysis.

A standard DNA sequencing apparatus (Owl Scientific S3S; Autogen Bioclear UK Ltd.) is used with a 60 sample comb (Owl Scientific S2S-60A; Autogen Bioclear UK Ltd.) and standard power supply (Biorad, Cat No. 165-5057) equipped with a temperature probe (Biorad, Cat No. 165-5058). A 0.4 mm thick 15% polyacrylamide gel is prepared using a 99:1 ratio of acrylamide to BAP cross linker, 10% ethylene glycol and 15% formamide in 0.5×TTE. Gels are pre-run for one hour at 30 watts, limiting the temperature to a maximum of 25ûC (using an electric fan to keep the temperature down if necessary eg. Jencons, Cat No. 292-004). After the pre-run, the wells are flushed with a pipette and the samples are loaded into the wells. The gel is then electrophoresed at 12 watts overnight (15 hours) at 25° C. Fragments greater than 350 bp remain on the gel.

After electrophoresis, the gel plates are separated. The gel is stained by placing the gel in 0.5×TTE containing lug/ml ethidium bromide (Biorad, Cat No. 161-0433) for 10 minutes, followed by destaining in 0.5×TTE for 10 minutes. The gel is then photographed on a UV transilluminator (eg. UVP GDS 7500).

Potential polynucleotide changes are detected by CSGE in five or more of the 16 patients for 9 of the 33 PCR fragments. For each of these potential changes, the PCR fragment from all 16 patients is subjected to double stranded DNA sequencing on an ABI377 automated sequencer using standard methods (http://www.pebio.com/ab/about/dna/377/) and the resulting DNA sequence is analysed using CONSED software (Gordon et al., Genome Res. 8:195-202) to confirm the presence of a sequence change and to identify the exact base change. All of the 9 potential changes detected by CSGE are confirmed. These changes are shown in the table below, in which #patients indicates the number of patients exhibiting the polymorphism:

| Alias | Polymorphism | # patients |
|---|---|---|
| Prom 1.3 | T to C | 8 |
| Prom 1.4 | A to C | 14 |
| Intron 1.1 | T to C | 14 |
| Intron 1.1 | G to A | 8 |
| Intron 1.3 | G to T | 13 |
| Intron 1.4 | A to G | 13 |
| Intron 1.4 | G to C | 13 |
| Exon 8.h | C to A | 7 |
| 3′.1 | C to T | 5 |

All nine polymorphisms are single nucleotide polymorphisms (SNPs). Several of the SNPs appear to be in linkage disequilibrium with each other as they occur with identical frequencies. In addition, the high frequencies also indicate a genetic association with the asthma phenotype in the patients and indicate that AAGB is an asthma susceptibility gene.

### EXAMPLE 3

This Example relates to the expression of full length AAGB with a 6 histidine tag at the C-terminus using the Baculovirus system in T.ni Hi5 cells, and to the purification of the resulting polypeptide.

1. Construction of a Recombinant AAGB Baculovirus

A unique EcoRI site is incorporated 5′ to the AAGB start codon by PCR amplification using the following primer:

5ST
5′-GAAGATCTTCGGAATTCCATCATGGCGTTGGCGTTGGCGGCGCTG

-3′

Another primer is used to introduce 6 histidine residues immediately prior to the AAGB stop codon. This primer also incorporates a unique KpnI site 3′ to the AAGB stop codon.

3ST
5′-AAGATCTTCGGTACCTTAATGGTGATGGTGATGGTGATAAATAAAGA
GAACTCTG-3′

The recombinant “His tagged” version of AAGB is ligated as a 694 bp EcoRI/KpnI fragment into EcoRI/KpnI digested pFastbacl baculovirus transfer vector (Life Sciences). The recombinant AAGB sequence is transposed into Bacmid DNA carried by DH10Bac cells (Life Sciences; Bac to Bac Baculovirus expression system). AAGB recombinant Bacmids are isolated from DHlOBac cells and transfected into Sf9 cells using published protocols (Bac to Bac baculovirus expression system manual; Life Sciences).

2. Amplification of Recombinant Baculovirus Stocks

The recombinant baculovirus is amplified by infecting Sf9 cells (maintained in SF900 SFMII medium; Life Sciences) at a cell density of 0.5×10⁶ cells/ml and a multiplicity of infection (mol) of 0.01 for 96 hours. Sf9 cells are then centrifuged at 1000×g for 5 minutes. The supernatants containing high titre virus are stored at 4° C.

3. Expression of Recombinant AAGB in Hi5 Cells

Hi5cells (Invitrogen), maintained at densities of between 3×10⁵ and 3×10⁶ cells/ml in Excell 401 medium (JRH Biosciences; distributed by AMS Biotechnology in either shaker flasks (rotated at 90 RPM) or spinner flasks (stirring at 75 RPM) are infected with the amplified recombinant Baculovirus at a cell density of 2.0×10⁶ at an moi of 2.0 for 60 hours. Following infection Hi5 cells are centrifuged at 1000×g for 5 minutes, the supernatants poured off and the cell pellets frozen at −80° C.

4. Crude Lysate Preparation

The cells (1×10⁹) are resuspended in 100 ml lysis buffer (20 mM Hepes pH 7.5, 100 mM NaCl, 5% glycerol, 2 mM beta-mercaptoethanol, 0.5 mM imidazole, 0.1% Nonidet P-40, 40 μg/ml AEBSF, 0.5 μg/ml leupeptin, 1 μg/ml aprotinin and 0.7 μg/ml pepstatin A). Cells are incubated on ice for 15 min then centrifuged at 39,000×g for 30 min at 4° C. The sample is filtered through a 0.22 μm filter immediately prior to use.

5. Metal Chelate Affinity Chromatography

Metal chelate affinity chromatography is carried out at room temperature with a column attached to a BioCAD chromatography workstation. A 20 ml Poros MC/M (16 mmD×100 mmL) column is charged with Ni²⁺ prior to use and after each injection. To charge with Ni²⁺, the column is washed with 10 column volumes (CV) 50 mM EDTA pH 8, 1 M NaCl followed by 10CV water. The column is charged with 500 ml 0.1 M NiSO4 pH 4.5-5, washed with 10CV water, then any unbound Ni²⁺ removed by washing with 5CV 0.3 M NaCl. All steps are performed with a flow rate of 20 ml/min. The charged MC/M column is equilibrated with 5CV Buffer B (20 mM Hepes pH 7.5, 500 mM NaCl, 5% glycerol, 2 mM β-mercaptoethanol, 1 mM PMSF, 5 mM imidazole) to saturate the sites followed by 10CV Buffer A (as Buffer B except 0.5 mM imidazole). 90-95 ml of the crude lysate is loaded onto the column per run at a flow rate of 20 ml/min. Subsequent steps are carried out with a flow rate of 30 ml/min. Any unbound material is removed by washing with 12 CV buffer A and AAGB eluted by applying a 0-50% Buffer B gradient over 10 CV. Fractions (8 ml) are collected over the gradient. AAGB-containing fractions are combined and protease inhibitors added to the final concentrations described for the lysis buffer above. DTT is also added to a final concentration of 1 mM. The combined fractions are dialysed overnight against 4 litres 20 mM Tris-HCl pH 7.5, 1 mM DTT, 0.2 mM PMSF at 4° C.

6. Ion Echange (Anion Exchange) Chromatography

Resource™ Q chromatography is carried out at 4° C. with a column attached to an FPLC workstation (Amersham Pharmacia Biotech). A 6 ml Resource™ Q column (16 mmD×30 mmL) is equilibrated with 10 CV Buffer C (20 mM Tris-HCl pH 7.5, 1 mM DTT) at a flow rate of 2 ml/min. The dialysed metal chelate eluate is applied to the column and washed with 10 CV Buffer C. The protein is eluted by applying a 0-100% Buffer D gradient (20 mM Tris-HCl pH 7.5, 1 mM DTT, 1 M NaCl) over 10 CV. Fractions (3 ml) are collected on eluting the column.

7. Gel Filtration

Del Filtration chromatography is carried out at 4° C. with a column attached to a BioCAD SPRINT chromatography workstation (PE Biosystems). A 318 ml (26 mmD×600 mmL) Sephacryl-200 prep (Amersham Pharmacia Biotech) column is equilibrated with 10 CV Buffer E (20 mM Tris-HCl pH7.5, 1 mM DTT, 150 mM NaCl) at a flow rate of 0.5 ml/min. The ion excahnge eluate is applied to the column and eluted with Buffer E. Fractions (1 ml) throughout the purification run are collected and analysed.

8. Sample Concentration

Samples are concentrated approximately 10-fold using a Millipore Ultrafree-15 centrifugation device (MW cut-off 50 kDa) at 4° C. The device is pre-rinsed with water prior to use. The final storage buffer used for long term storage at −80° C. is 20 mM Hepes pH 7.5, 1 mM DTT, 100 mM NaCl, 5% glycerol. Glycerol can be omitted from the sample for storage at 4° C.

### EXAMPLE 4

This example relates to the production of polyclonal antibodies against AAGB protein purified as described in Example 3. Polyclonal antibodies against a recombinant fragment spanning the extracellular domain are generated as described by Telo et al. (J. Biol. Chem. 273:17565-17572).

Immunisation of Rabbits

Dutch rabbits (Harlen-Olac) are immunised at 4 subcutaneous sites with 500 μg purified AAGB protein in PBS according to the following schedule:

| DAYS | IMMUNISATIONS |
|---|---|
| 0 | 1^{st} immunisation 1:1 in complete Freund's adjuvant |
| 15 | 1^{st} boost 1:1 in incomplete Freund's adjuvant |
| 45 | 2^{nd} boost 1:1 in incomplete Freund's adjuvant |
| 55 | 1^{st} test bleed from the ear artery |
| Every month | Boost 1:1 in incomplete Freund's adjuvant until a |
| | good antibody response is obtained |

Test bleeds (500 μl) are taken and the serum assessed for antibody titre. Serum is collected when a maximum titre is reached. This is done by collecting blood (10 ml) and allowing it to clot for 2 hours at 4° C. The blood is centrifuged at 1000×g for 5 minutes to separate the serum. The serum is removed and stored at −20° C. until assayed.

ELISA Screening

Nunc-Immuno Plate Maxisorp 96 well plates (Nunc, Fisher Scientific UK, Loughborough, UK) are used as a solid support and coated with the purified AAGB protein (100 ng/well) overnight at 4° C. The plates are blocked for 3 hours at 37° C. with PBS containing 2% BSA (Sigma) and 0.02% NaN₃ (Sigma). After blocking, plates are incubated overnight at room temperature with serum in different dilutions of PBS. The presence of polyclonal5 antibodies is checked with both biotin labelled IgG-antibodies to rabbit (Goat anti-rabbit IgG antiserum, 1:25000 dilution), with an incubation time of 40 min. Alkaline phosphatase conjugated streptavidin (Immununo Research, Dianova, CH) is then added at a dilution of 1:10000. Development of the reaction is carried out by adding an alkaline phosphatase substrate (Sigma, f.c. 1 mg/ml) dissolved in diethanolamine. After 45 min. absorbance is read at 405 nm with a reference of 490 nm with an ELISA plate reader (Bio-rad laboratories Ltd., Hemel Hempstead, UK).

Purification

5 ml protein A-agarose is poured into a chromatography column and washed with 6 column volumes of 0.1 M tris (hydroxymethyl) methylamine (Tris) buffer pH 7.5. The rabbit serum containing anti-AAGB antibodies is diluted (½) with Tris buffer and added to the protein A-agarose. Unbound proteins are removed by washing the column with 6 volumes of Tris buffer. The IgG is eluted off the column with three column volumes of 0.1 M glycine buffer pH 3.0 and collected as 1 ml fractions into tubes containing 28 μl of 1 M Tris. The fractions which are positive for protein content are checked for purity by SDS-PAGE under reducing conditions. Two bands at 50 and 25 Kd are visualised corresponding to the heavy and light chains of an immunoglobulin molecule. Fractions containing only immunoglobulin are pooled, re-checked for protein concentration and stored at −20° C.

### EXAMPLE 5

This exaple describes the preparation of monoclonal antibodies against AAGB protein purified as described in Example 3.

Immunisation od Mice

Female Balb/c mice are immunised intraperitoneally with 100 μg of AAGB protein in PBS according to the given below:

| DAYS | IMMUNISATIONS |
|---|---|
| 1 | 1^{st} immunisation 1:1 with complete |
| | Freund's adjuvant |
| 14 | 1^{st} boost 1:1 with incomplete Freund's |
| | adjuvant |
| 21 | 2^{nd} boost 1:1 with incomplete Freund's |
| | adjuvant |
| 28-30 | Three final boosts in PBS |
| 31 | Fusion with mouse myeloma cells |

Serum is assessed for antibody titre by ELISA (Example 4) after the animal is sacrificed for the preparation of spleen cells for fusion. If antibody titre is sufficient, (1/1000 to 1/100,000), the hybridomas are screened, otherwise discarded.

Preparation of Myeloma Cells

Sp2/0 murine myeloma cells (ATCC #CRL 1581; maintained in culture medium containing 20 μg/ml 8-azaguanine) are cultivated for one week before fusion in RPMI 1640 (8-azaguanine is not included), 10% (v/v) FCS and 1% penicillin-streptomycin (50IU/ml and 50 μg/ml, respectively). The cells are harvested by centrifugation (200×g for 5 min) and washed three times in cold RPMI 1640. Approximately 2.5×10⁶ cells are used per 96 well microtitre plate.

Preparation of Spleen Cell Suspension

The mouse is killed by an overdose of anesthetic (Forene), the spleen dissected and pressed through a cell strainer (70 μm mesh cell strainer; Becton & Dickinson, Oxford, UK, Cat. No 2350). The cell suspension is washed three times in RPMI 1640 (as above) and counted: 5.10⁶ cells /96 well plate are necessary.

Fusion of Myeloma Cells and Spleen Cells

The spleen and myeloma cells are mixed (2:1), centrifuged (200 ×g for 5 min) and the pellet warmed in a 37° C. water bath. Prewarmed polyethylene glycol 4000 (1 ml per 10⁸ cells) is added slowly over one minute, then 20 ml of prewarmed wash medium over two minutes. After centrifugation the pellet is carefully resuspended in selection medium (RPMI 1640, 10% FCS, 1% penicillin-streptomycin, 10% BM condimed H1 (feeder cell replacement from Boehringer Mannheim, Lewes, UK; Cat. No. 1 088 947), 10% HAT-media supplement (hypoxanthine, aminopterin and thymidine to select against unfused myeloma cells; Boehringer Mannheim, Lewes, UK; Cat. No. 644 579) and plated, 200 μl/well of a 96 well microtitre plate.

After five days clusters of hybrid cells can be identified by examining the bottom of the microtitre wells with an inverted microscope. After 10-14 days the culture supernatant is tested for the presence of antibodies by ELISA (example 4). The positive clones are expanded in a 24 well assay plate and retested.

Cloning of Positive Hybridomas

The expanded clones which are still positive are cloned by limiting dilution. Cells are diluted serially in four dilutions steps in a 96 well microtitre plate; 5, 2, 1 and 0.5 cells/well. HAT-media supplement is replaced with HT-media supplement (Boehringer Mannheim, Lewes, UK; Cat. No. 623 091). After approximately one week the cells are screened by ELISA (Example 4). The cells of those wells containing a single positive clone are expanded.

Production of Monoclonal Antibody Supernatant

The cells are grown in culture flasks in standard medium (RPMI 1640, 10% (v/v) FCS and 1% penicillin-streptomycin) until the hybridomas overgrow and die. The debris is removed by centrifugation and the supernatant containing the antibodies is titred using ELISA (Example 4) before storing under sterile conditions at 4° C., −20° C. or −70° C.

What is claimed is:

1. An isolated polynucleotide comprising a nucleotide sequence encoding a polypeptide comprising the amino acid sequence of SEQ ID NO:4 or a functionally equivalent variant of said amino acid sequence.

2. An isolated polynucleotide according to claim 1 which is cDNA comprising the nucleotide sequence of SEQ ID NO:1 or SEQ ID NO:2 or SEQ ID NO:3 or SEQ ID NO:5 or a DNA comprising a nucleotide sequence which hybridises to SEQ ID NO:1 or SEQ ID NO:2 or SEQ ID NO:3 or SEQ ID NO:5 under stringent conditions.

3. An isolated polynucleotide according to claim 1 which is a genomic DNA comprising the nucleotide sequence of SEQ ID NO:6 or SEQ ID NO:7 or a DNA comprising a nucleotide sequence which hybridises to SEQ ID NO:6 or SEQ ID NO:7 under stringent conditions.

4. An isolated polynucleotide comprising a consecutive 20 base pair nucleotide portion identical in sequence to a consecutive 20 base pair portion of SEQ ID NO:1 or SEQ ID NO:2 or SEQ ID NO:3 or SEQ ID NO:5 or SEQ ID NO:6 or SEQ ID NO:7.

5. An isolated polypeptide comprising the amino acid sequence of SEQ ID NO:4 or a functionally equivalent variant thereof.

6. An isolated polypeptide comprising a consecutive 10 amino acid portion identical in sequence to a consecutive 10 amino acid portion of SEQ ID NO:4.

7. A method of producing a polypeptide according to claim 5 which comprises culturing a host cell containing an expression vector containing a polynucleotide sequence as specified in claim 1, under conditions suitable for expression of the polypeptide and recovering the polypeptide from the host cell culture.

8. An isolated polypeptide encoded by a variant of the nucleotide sequence specified in claim 1 having a sequence polymorphism correlated with a disease.

9. An antibody which is immunoreactive with a polypeptide comprising SEQ ID NO:4 or a polypeptide according to claim 8.

10. An antisense oligonucleotide comprising a nucleotide sequence complementary to that of a polynucleotide encoding a polypeptide according to claim 5 or a variant thereof having a polymorphism correlated with a disease.

11. A polynucleotide probe comprising at least 15 contiguous nucleotides of a polynucleotide according to claim 1, or a complement thereof.

12. A pharmaceutical composition comprising a polypeptide (A) comprising the amino acid sequence of SEQ ID NO:4 or a functionally equivalent variant thereof, a polynucleotide comprising a nucleotide sequence encoding said polypeptide (A), an antibody which is immunoreactive with said polypeptide (A) or a polypeptide according to claim 8 or an antisense oligonucleotide comprising a nucleotide sequence complementary to that of said polynucleotide or a variant thereof having a polymorphism correlated with a disease, optionally together with a pharmaceutically acceptable carrier.

13. A method of treating an inflammatory or obstructive airways disease which comprises administering to a subject in need thereof an effective amount of a polypeptide (A) comprising the amino acid sequence of SEQ ID NO:4 or a functionally equivalent variant thereof, a polynucleotide comprising a nucleotide sequence encoding said polypeptide (A), an antibody which is immunoreactive with said polypeptide (A) or a polypeptide according to claim 8, or an antisense oligonucleotide comprising a nucleotide sequence complementary to that of said polynucleotide or a variant thereof having a polymorphism correlated with a disease.

14. A method of detecting genetic abnormality in a subject which comprises incubating a genetic sample from the subject with a polynucleotide probe according to claim 11, under conditions where the probe hybridises to complementary polynucleotide sequence, to produce a first reaction product, and comparing the first reaction product to a control reaction product obtained with a normal genetic sample, where a difference between the first reaction product and the control reaction product indicates a genetic abnormality in the subject or a predisposition to developing a disease.

15. A method of detecting the presence of a polynucleotide according to claim 1 in a cell or tissue which comprises contacting DNA from the cell or tissue with a polynucleotide probe comprising at least 15 contiguous nucleotides of a polynucleotide according to claim 1 under conditions where the probe is specifically hybridizable with a polynucleotide according to claim 1, and detecting whether hybridization occurs.

16. A method of detecting an abnormality in the nucleotide sequence of a polynucleotide according to claim 1 in a patient which comprises amplifying a target nucleotide sequence, in DNA isolated from the patient, by a polymerase chain reaction using a pair of primers which target the sequence to be amplified and analysing the amplified sequence to determine any polymorphism present therein.

17. A pair of oligonucleotides useful as primers for amplification of a fragment of a polynucleotide according to claim 1, each oligonucleotide of said pair being at least 15 nucleotides in length and said pair having sequences such that when used in a polymerase chain reaction with human genomic DNA or a suitable human cDNA target, they result in synthesis of a DNA fragment containing part or all of the nucleotide sequence of a polynucleotide according to claim 1.

18. A variant of a polynucleotide comprising a nucleotide sequence encoding a polypeptide comprising amino acid sequence SEQ ID NO:4, which variant contains a sequence polymorphism correlated with asthma.

19. A method of determining predisposition of a patient to asthma which comprises identifying in DNA from the patient a sequence polymorphism or haplotype in a nucleotide sequence encoding a polypeptide comprising amino acid SEQ ID NO:4, as compared with a normal control DNA from a non-asthmatic subject, which correlates with asthma.

20. A method of identifying a substance which modulates the activity of a polypeptide comprising amino acid sequence SEQ ID NO:4 or a functionally equivalent variant thereof or a polypeptide according to claim 8 comprising combining a candidate substance with said polypeptide and measuring the effect of the candidate substance on said activity.
